(19) United States
(12) Patent Application Publication
(10) Pub. No.: US 2022/0105176 A1
(43) Pub. Date: Apr. 7, 2022

(54) POLYMERSOMES COMPRISING A COVALENTLY BOUND ANTIGEN AS WELL AS METHODS OF MAKING AND USES THEREOF

(71) Applicant: ACM BIOLABS PTE LTD, Singapore (SG)

(72) Inventors: Madhavan NALLANI, Singapore (SG); Fabien DECAILLOT, Singapore (SG); Thomas Andrew CORNELL, Singapore (SG); Amit Kumar KHAN, Singapore (SG)

(73) Assignee: ACM BIOLABS PTE LTD, Singapore (SG)

(21) Appl. No.: 17/276,023

(22) PCT Filed: Sep. 12, 2019
(86) PCT No.: PCT/EP2019/074308

§ 371 Date: Mar. 12, 2021

(30) Foreign Application Priority Data
Sep. 12, 2018 (EP) 18193946.3

(51) Int. Cl. A61K 39/39; A61K 39/12; C12N 7/00

(52) U.S. Cl. CPC A61K 39/39; A61K 2039/55555; C12N 7/00; A61K 39/12

(57) ABSTRACT

The present invention relates to polymersomes capable of eliciting an immune response, comprising: an antigen selected from the group consisting of: (a) a polypeptide; (b) a carbohydrate; (c) a polynucleotide; and (d) a combination of (a) and/or (b) and/or (c); wherein the antigen is conjugated to the exterior surface of the polymersome via a covalent bond. The present invention further relates to a method for production of antigens conjugated to a polymersome as well as to polymersomes produced by said method. The present invention further relates to compositions comprising a polymersome of the present invention, isolated antigen presenting cells or hybridoma cells exposed to the polymersome or composition of the present invention. The present invention also relates to vaccines comprising polymersomes of the present invention, methods of eliciting an immune response or methods for treatment, amelioration, prophylaxis or diagnostics of a cancer, autoimmune or infectious disease, comprising providing polymersomes of the present invention.

### CROSS-REFERENCE TO RELATED APPLICATIONS

This application claims the benefit of priority of European patent application no. 18193946.3 filed 12 Sep. 2018, the contents of which is being hereby incorporated by reference it its entirety for all purposes.

### SEQUENCE LISTING

This application contains a Sequence Listing in computer readable form, which is incorporated herein by reference.

### TECHNICAL FIELD

The present invention relates to polymersomes capable of eliciting an immune response, comprising: an antigen selected from the group consisting of: (a) a polypeptide; (b) a carbohydrate; (c) a polynucleotide; and (d) a combination of (a) and/or (b) and/or (c); wherein the antigen is conjugated to the exterior surface of the polymersome via a covalent bond. The present invention further relates to a method for production of antigens conjugated to a polymersome as well as to polymersomes produced by said method. The present invention further relates to compositions comprising a polymersome of the present invention, isolated antigen presenting cells or hybridoma cells exposed to the polymersome or composition of the present invention. The present invention also relates to vaccines comprising polymersomes of the present invention, methods of eliciting an immune response or methods for treatment, amelioration, prophylaxis or diagnostics of a cancer, autoimmune or infectious disease, comprising providing polymersomes of the present invention.

### BACKGROUND OF THE INVENTION

Although immunization is a well-established process, there are differences in the response level elicited between different immunogens or antigens. For example, membrane proteins form a class of antigens that produce a low response level, which in turn means that large amounts of membrane proteins are required to generate or elicit an immune response to the desired level. Membrane proteins are notoriously difficult to synthesize and are insoluble in water without the presence of a detergent. This makes it expensive and difficult to obtain membrane proteins in sufficient quantity for immunization. Furthermore, membrane proteins require proper folding to function correctly. The immunogenicity of correctly folded native membrane proteins is typically much better than that of their solubilized forms, which may not be folded in a physiologically relevant manner. Thus, even though adjuvants may be used to boost the immunogenicity of such solubilized antigens, it is an inefficient method that does not provide too much of an advantage (e.g., WO2014/077781A1).

Although transfected cells and lipid-based systems have been used to present membrane protein antigens to increase the chances of isolating antibodies that may be efficient in vivo, these systems are often unstable (e.g., oxidation sensitive), tedious and costly. Moreover, the current state of the art for such membrane protein antigens is to use inactive virus-like particles for immunization.

On the other hand, vaccines are the most efficient way to prevent diseases, mainly infectious diseases [e.g., Liu et al., 2016]. As of today, most of the licensed vaccines are made of either live or killed viruses. Despite their effectiveness in generating a humoral response (an antibody mediated response) to prevent viral propagation and entry into cells, safety of such vaccines remains a concern. In the past few decades, scientific advances have helped to overcome such issues by engineering vaccine vectors that are non-replicating recombinant viruses. In parallel, protein-based antigens or sub-unit antigens are explored as safer alternatives. However, such protein-based vaccines typically illicit poor immune (both humoral and cellular response). To improve immunogenic properties of antigens, several approaches have been used. For example, microencapsulation of antigens into polymers have been investigated extensively, although it did enhance the immunogenicity, aggregation and denaturing of antigens remain unsolved [e.g., Hilbert et al., 1999]. Furthermore, adjuvants (e.g., oil in water emulsions or polymer emulsions) [e.g., U.S. Pat. No. 9,636,397B2, US2015/0044242 A1] are used together with antigens to elicit a more pronounced humoral and cellular response. Despite these advances, they are less efficient in uptake and cross-presentation. To promote cross-presentation, based on the available information of the immune system during infection by viruses, viral like particles that mimics such properties have been exploited. Synthetic architectures such as liposomes with encapsulated antigens are particularly attractive. Liposomes are unilamellar self-assembling structures made of lipids and, cationic liposomes are more attractive and promising as delivery vehicles because of their efficient uptake by Antigen Presenting Cells (APCs) [e.g., Maji et al., 2016]. Furthermore, it allows to integrate immunomodulators such as Monophosphoryl Lipid A (MPL), CpG oligodeoxynucleotide, that are toll-like receptor (TLR) agonists which stimulate immune cells through receptors. Despite these opportunities of such delivery vehicles, one of the limiting factors is stability of liposomes in the presence of serum components. By PEGlyations, loading with high melting temperature lipids, stability issues of liposomes are somewhat reduced with and one such well characterized example being interbilayered-crosslinked multilamellar vesicles (ICMVs), formed by stabilizing multilamellar vesicles with short covalent crosslinks linking lipids [e.g., Moon et al., 2011]. Other nanoparticle architectures have led to successful immunisations using nanodiscs [e.g., Kuai et al., 2017] or pH sensitive particles [e.g., Luo et al., 2017]. But such strategies are not yet demonstrated successfully in clinics owing to the unstable properties of lipid associated carriers.

Therefore, there remains a need to provide for improvement of immunogenic properties of antigens, efficient uptake and stable cross-presentation delivery vehicles and methods that overcome, or at least alleviate, the above problems as well as possess an improved functionality inter alia in that they are also capable of eliciting an immune response (e.g., humoral immune response), which is particularly important in treatment and/or prevention of infectious diseases, cancers and autoimmune diseases.

Polymersomes, on the other hands offer as a stable alternative for liposomes and they have been used to integrate membrane proteins to elicit immune response [e.g., Quer et al., 2011, WO2014/077781A1]. Protein antigens were also encapsulated in a chemically altered membrane of the polymersome (however oxidation-sensitive membranes) to release antigens and the adjuvants to dendritic cells [e.g., Stano et al., 2013].

In the course of the present invention, polymersomes were employed that were made of amphiphilic block copolymers without any known characteristic, with antigens conjugated to the exterior surface of said polymersomes via a covalent bond, as better delivery vehicles to the immune system (i.e., without any chemical modification to favour release) inter alia in order to enhance the immune response and elicit a strong humoral response. As model antigens, Ovalbumin (OVA) and Influenza Hemagglutinin (HA) were used in such polymersomes and a better humoral immune response in comparison to protocols based on, e.g., immunostimulant molecules such as Sigma Adjuvant System (SAS) or encapsulated antigens, has been demonstrated.

Therefore, the present invention inter alia provides polymersomes of the present invention as efficient uptake and stable cross-presentation delivery vehicles improving immunogenic properties of antigens and methods based thereon, e.g., in order to present soluble or solubilized antigens (or soluble or solubilized portions thereof) conjugated to the exterior surface of the polymersome via a covalent bond to the immune system and evoke an immune response comprising a strong titers of specific antibodies, e.g., without addition of known adjuvants.

### SUMMARY OF THE INVENTION

The present invention relates to a polymersome capable of eliciting an immune response, comprising: an antigen selected from the group consisting of: (a) polypeptide, (b) a carbohydrate, (c) a polynucleotide, (d) a combination of (a) and/or (b) and/or (c), wherein the antigen is conjugated to the exterior surface of the polymersome via a covalent bond.

Furthermore, in the course of the present invention it was found that providing the polymersomes of the present invention (also referred to as “ACMs” herein) allows, for example, soluble (or solubilized) antigens conjugated to the exterior surface of said polymersomes via a covalent bond to produce a stronger humoral immune response (compared to free antigens with or without adjuvants as well as encapsulated antigens). Consequently, an increase in the efficiency of antibody production in a subject is achieved. The increase in the efficiency can be attained with or without the use of adjuvants. Furthermore, the ability of the polymersomes of the present invention to elicit a CD8(+) T cell-mediated immune response dramatically increases their potential as an immunotherapeutic antigen delivery and presentation system.

Because soluble (e.g., solubilized) antigens conjugated to the exterior surface of polymersomes of the present invention via a covalent bond have improved immunogenic properties, the antibodies produced by the use of such polymersomes and methods based thereon would not only have a higher production success rate and higher affinity for their corresponding in vitro or in vivo targets and accordingly improved sensitivity when used in various solution-based antibody applications, but also would make possible to easily raise antibodies to difficult antigens not capable of triggering antibody production by conventional methods using free antigen injections and/or decrease the amount of antigen required for such antibody production procedure thus decreasing the cost of such a production. Furthermore, soluble (e.g., solubilized) antigens conjugated to the exterior surface of polymersomes of the present invention are also capable of eliciting a CD8(+) T cell-mediated immune response, which extends the use of corresponding polymersomes to cell-mediated immunity and therefore improves their immunotherapeutic- and antigen delivery and presentation potential.

Therefore, the present application satisfies this demand by provision of polymersomes capable of eliciting an immune response, comprising: an antigen selected from the group consisting of: (a) polypeptide, (b) a carbohydrate, (c) a polynucleotide, (d) a combination of (a) and/or (b) and/or (c), wherein the antigen is conjugated to the exterior surface of the polymersome via a covalent bond, that improve immunogenic properties of antigens, methods for their production and compositions comprising such polymersomes, described herein below, characterized in the claims and illustrated by the appended Examples and Figures.

### Overview of the Sequence Listing

As described herein references are made to UniProtKB Accession Numbers (http://www.uniprot.org/ e.g., as available in UniProtKB Release 2017_12)
SEQ ID NO: 1 is the amino acid sequence of the chicken Ovalbumin (OVA), UniProtKB Accession Number: P01012.
SEQ ID NO: 2 is the amino acid sequence of the influenza A virus (A/New York/38/2016(H1N1)) hemagglutinin, UniProtKB Accession Number: A0A192ZYK0.
SEQ ID NO: 3 is the amino acid sequence of the influenza A virus (A/swine/4/Mexico/2009(H1N1)) hemagglutinin, UniProtKB Accession Number: D2CE65.
SEQ ID NO: 4 is the amino acid sequence of the influenza A virus (A/Puerto rico/8/1934(H1N1)) hemagglutinin.
SEQ ID NO: 5 is the amino acid sequence of the influenza A virus (A/California/07/2009(H1N1)) hemagglutinin.

### BRIEF DESCRIPTION OF THE DRAWINGS

FIG. 1: Dynamic Light Scattering (DLS) spectra of OVA conjugated ACMs.

FIG. 2: Characterization of OVA conjugated ACMs. (A) SEC profile of OVA conjugated ACMs. (B) SDS-PAGE loaded with samples from SEC peak and stained using silver staining.

FIG. 3: DLS spectra of HA conjugated ACMs.

FIG. 4: Immunoblot of ACM conjugated HA samples. Coupled and free HA migrate differently.

FIG. 5: SEC profile of HA conjugated ACMs (mAU, light gray trace) superimposed with ELISA signals performed on all collected fractions (O.D. 450, black trace).

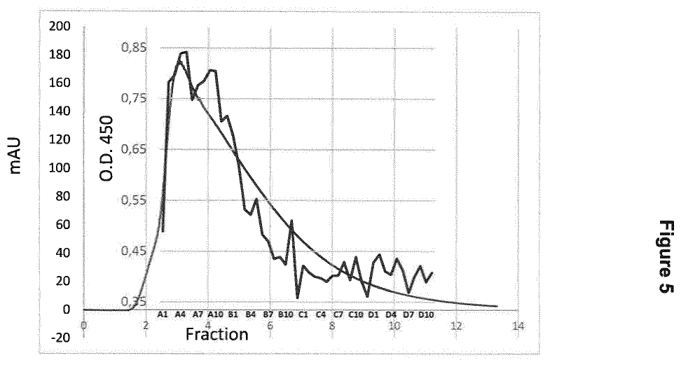

FIG. 6: Antibody titers from sera of immunized C57BI/6 mice with PBS, free OVA, free OVA with SAS, BD21 encapsulated OVA and BD21 conjugated OVA, p<0.01.

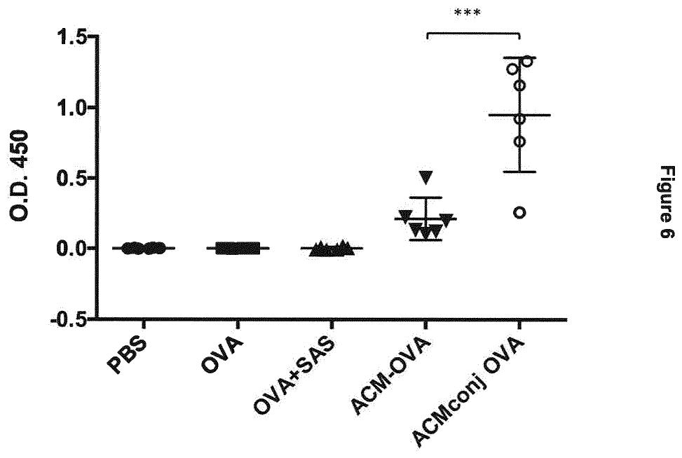

FIG. 7: Antibody titers from sera of immunized Balb/c mice with PBS, free HA, BD21 encapsulated HA and BD21 conjugated HA.

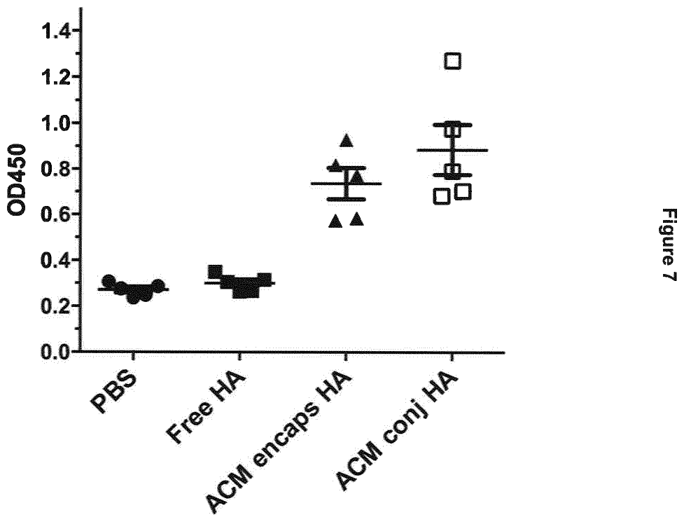

### DETAILED DESCRIPTION OF THE INVENTION

The following detailed description refers to the accompanying Examples and Figures that show, by way of illustration, specific details and embodiments, in which the invention may be practised. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

In the present context, polymersomes (also referred to as “ACMs” herein) are vesicles with a polymeric membrane, which are typically, but not necessarily, formed from the self-assembly of dilute solutions of amphiphilic block copolymers, which can be of different types such as diblock and triblock (A-B-A or A-B-C). Polymersomes of the present invention may also be formed of tetra-block or penta-block copolymers. For tri-block copolymers, the central block is often shielded from the environment by its flanking blocks, while di-block copolymers self-assemble into bilayers, placing two hydrophobic blocks tail-to-tail, much to the same effect. In most cases, the vesicular membrane has an insoluble middle layer and soluble outer layers. The driving force for polymersome formation by self-assembly is considered to be the microphase separation of the insoluble blocks, which tend to associate in order to shield themselves from contact with water. Polymersomes possess of the present invention remarkable properties due to the large molecular weight of the constituent copolymers. Vesicle formation is favored upon an increase in total molecular weight of the block copolymers. As a consequence, diffusion of the (polymeric) amphiphiles in these vesicles is very low compared to vesicles formed by lipids and surfactants. Owing to this less mobility of polymer chains aggregated in vesicle structure, it is possible to obtain stable polymersome morphologies. Unless expressly stated otherwise, the term “polymersome” and “vesicle”, as used herein, are taken to be analogous and may be used interchangeably. In some aspects, the polymersome of the present invention is oxidation-stable.

In some aspects, the present invention relates to a polymersome capable of eliciting an immune response, comprising: an antigen selected from the group consisting of: (a) polypeptide, (b) a carbohydrate, (c) a polynucleotide, (d) a combination of (a) and/or (b) and/or (c), wherein the antigen is conjugated to the exterior surface of the polymersome via a covalent bond. According to the present invention said covalent bond can be any suitable covalent bond capable of conjugating an antigen (e.g., the antigen of the present invention) to the exterior surface of the polymersome of the present invention.

In some aspects, the present invention relates to a method for eliciting an immune response to a soluble (e.g., solubilized) antigen conjugated to the exterior surface of said polymersomes via a covalent bond and/or encapsulated antigen in a subject. The method is suitable for administering to the subject, for example, orally or by injection, a composition comprising a polymersome (e.g., carrier or vehicle) having a membrane (e.g., circumferential membrane) of an amphiphilic polymer. The composition of the present invention comprises a soluble (e.g., solubilized) antigen conjugated to the membrane (e.g., circumferential membrane) of the amphiphilic polymer of the polymersome of the present invention.

The antigen of the present invention may be one or more of the following: i) a polypeptide (e.g., including short peptides of up to 10 amino acids or also longer peptides with more than 10 amino acid residues, e.g., tumour neoantigen polypeptides); ii) a carbohydrate; iii) a polynucleotide (e.g., said polynucleotide is not an antisense oligonucleotide, preferably said polynucleotide is a DNA or messenger RNA (mRNA) molecule) or a combination of i) and/or ii) and/or iii).

According to the present invention said covalent bond can be any suitable covalent bond capable of conjugating an antigen (e.g., the antigen of the present invention) to the exterior surface of the polymersome of the present invention. Conjugating reactions producing covalent bonds of the present invention are well known in the art (e.g., NHS-EDC conjugations, reductive amination conjugations, sulfhydryl conjugations, “click” and “photo-click” conjugations, pyrazoline conjugations etc.). Non-limiting examples of such covalent bonds and methods of producing thereof are listed below herein. Thus, in some aspects, the covalent bond via which the antigen of the present invention is conjugated to the exterior surface of the polymersome of the present invention comprises: i) an amide moiety (e.g., as described in the Examples section herein); and/or ii) a secondary amine moiety (e.g., as described in the Examples section herein); and/or iii) a 1,2,3-triazole moiety (e.g., as described in van Dongen et al., A Block Copolymer for Functionalisation of Polymersome Surfaces, Macromolecular Rapid Communication 2008, 29, 321-325), preferably said 1,2,3-triazole moiety is a 1,4-disubstituted[1,2,3]triazole moiety or a 1,5-disubstituted[1,2,3]triazole moiety (e.g., as described in Boren et al., Ruthenium-catalyzed azide-alkyne cycloaddition: scope and mechanism. J Am Chem Soc. 2008 Jul. 16; 130(28):8923-30. doi: 10.1021/ja0749993. Epub 2008 Jun. 21); and/or iv) pyrazoline moiety (e.g., as described in de Hoog et al., A facile and fast method for the functionalization of polymersomes by photoinduced cycloaddition chemistry, Polym. Chem., 2012, 3, 302-306), and/or an ether moiety. It is noted in this context that it might be necessary to modify both the polymersome and the antigen, for example a protein, for the conjugation/formation of the covalent bond between the exterior surface of the polymersome and the antigen. In addition to classical chemical conjugation chemistry (reaction) as described above, it is also possible to form the covalent bond between the exterior surface of the polymersome and the antigen by enzymatic reaction.

In some aspects, the present invention relates to NHS-EDC conjugation (i.e., conjugation based on N-hydroxysuccinimide (NHS), and 1-Ethyl-3-(3-dimethylaminopropyl)-carbodiimide (EDC)) is one of the exemplary alternative ways of conjugating antigens to polymersomes of the present invention. In this method, carboxylic acid groups react with EDC producing an intermediate O-acylisourea that is then reacts with primary amines to form an amide moiety with said carboxyl group.

In some aspects, the present invention relates to a reductive amination conjugation, which is another exemplary alternative way of conjugating antigens to polymersomes of the present invention. In this method an aldehyde-containing compound is conjugated to amine-containing compound to form a Schiff-base intermediate that in turn undergoes reduction to form a stable secondary amine moiety.

In some aspects, the present invention relates to a sulfhydryl conjugation, which is another exemplary alternative way of conjugating antigens to polymersomes of the present invention. In this method sulfhydryl (—SH) containing compound (e.g., present in side chains of cysteine) is conjugated to sulfhydryl-reactive chemical group (e.g., maleimide) via alkylation or disulfide exchange to form a thioether bond or disulfide bond respectively.

In some aspects, the present invention relates to a so-called “click” reaction (also known as “azide-alkyne cycloaddition”) on polymersome surface (e.g., described by van Dongen et al., Macromol. Rapid Communications, 2008, 29, pages 321-325), which is another exemplary alternative way of conjugating antigens to polymersomes of the present invention. According to this method a 1,2,3-triazole moiety is produced in that an aqueous solution of azido-functionalised antigens (e.g., a polypeptide) is added to a dispersion of polymersomes, followed by an addition of a premixed aqueous solutions of Cu(II)SO₄.5H₂O with sodium ascorbate and bathophenanthroline ligand to the resulting dispersion of polymersomes and then left at 4° C. for 60 hours, followed by filtering of said dispersion with a 100 nm cutoff and centrifuging to dryness. In this context it is further noted that copper-catalysed reaction of azide-alkyne cycloaddition” (also known as CuAAC) allows for synthesis of the 1,4-disubstituted regioisomers specifically, whereas a ruthenium-catalysed reaction of azide-alkyne cycloaddition (also known as RuAAC) (e.g., using Cp*RuCl(PPh₃)₂ as catalysator) allows for the production of 1,5-disubstituted triazoles (cf. Johansson et al., Ruthenium-Catalyzed Azide Alkyne Cycloaddition Reaction: Scope, Mechanism, and Applications. Chem Rev. 2016 Dec. 14; 116(23):14726-14768. Epub 2016 Nov. 18. 2016).

In some aspects, the present invention relates to a photo-induced generation of the nitrile imine intermediate (e.g., generated from bisaryl-tetrazoles) and its cycloaddition to alkenes (a so-called photo-induced cycloaddition or “photo-click” reaction, e.g., described by de Hoog et al., supra 2012), which is another exemplary alternative way of conjugating antigens to polymersomes of the present invention. According to this method, ABA block copolymer is methacrylate (MA) terminated or hydroxyl terminated with tetrazole by the photo-induced generation of the nitrile imine intermediate producing ABA polymersomes containing MA-ABA and hydroxyl terminated ABA copolymer, followed by reacting said polymersomes with tetrazole-containing antigen (HRP) under UV-irradiation to produce a pyrazoline moiety.

The covalent bond that conjugates the antigen to the exterior surface of the polymersome can either be formed between an atom/group of a molecule such an amphilic polymer that is part of (present in) of the circumferential membrane of the polymersome. Alternatively, the covalent bond between the antigen and the exterior surface of the polymer is formed via a linker moiety that is attached to a molecule that that is part of (present in) of the circumferential membrane of the polymersome. The linker may have any suitable length and can have a length of one main chain atom (for example, if the linker is a simple carbonyl group (C═O) that yields an amide or an ester moiety forming the covalent linkage). An illustrative example for such “one atom/linker moiety with a length of one main atom is the modification of the amphilic polymer BD21 by Dess-Martin periodinane carried out in the Example Section to yield BD₂₁-CHO (i.e. a terminal aldehyde group) which is then used to form an amine bond with the selected antigen (hemagglutinin is used as a purely illustrative example antigen in the Experimental Section. Alternatively, the linker moiety may have a length of several hundreds or even more main chain atoms, for example, if a moiety such as polyethylenglycol (PEG) that is commonly used for conjugation (convalent coupling) of polypeptides with a molecule of interest. As a purely illustrative example see distearoylphosphatidylethanolamine [DSPE] polyethylene glycol (DSPE-PEG) conjugates discussed below and used in the Example Section of the present application. The DSPE-PEG(3000) linker moiety used in the Example section has about 65 ethylene oxide (CH₂—CH₂—O)-subunit and thus about 325 main chain atom in the PEG part alone and a total length of about 408 main chain atoms. In line with the above, illustrative embodiments, the linker moiety may comprise 1 to about 550 main chain atoms, 1 to about 500 main chain atoms, 1 to about 450 main chain atoms, 1 to about 350 main chain atoms, 1 to about 300 main chain atoms, 1 to about 250 main chain atoms, 1 to about 200 main chain atoms, 1 to about 150 main chain atoms, 1 to about 100 main chain atoms, 1 to about 50 main chain atoms, 1 to about 30 main chain atoms, 1 to about 20 main chain atoms, 1 to about 15 main chain atoms, or 1 to about 12 main chain atoms, or 1 to about 10 main chain atoms, wherein the main chain atoms are carbon atoms that are optionally replaced by one or more heteroatoms selected from the group consisting of N, O, P and S.

Also in accordance with the above disclosure, the linker moiety may be a peptidic linker or a straight or branched hydrocarbon-based linker. The linker moiety may also be or a co polymer with a different block length. The linker moiety used in the present invention may comprise a membrane anchoring domain which integrates the linker moiety into the membrane of the polymersome. Such a membrane anchoring domain may comprise a lipid such as a phospholipid or a glycolipid. The glycolipid used in membrane anchoring domain may comprise glycophosphatidylinositol (GPI) which has been widely used a membrane anchoring domain (see, for example, International Patent Applications WO 2009/127537 and WO 2014/057128). The phospholipid used in the linker of the present invention may be phosphosphingolipid or a glycerophospholipid. In illustrative examples of such a linker, the phosphosphingolipid may comprise as a membrane anchoring domain distearoylphosphatidylethanolamine [DSPE] conjugate to polyethylene glycol (PEG) (DSPE-PEG). In such conjugates, the DSPE-PEG may comprise any suitable number of ethylene oxide, for example, from 2 to about 500 ethylene oxide units. Illustrative examples include DSPE-PEG(1000), DSPE-PEG(2000) or DSPE-PEG(3000) to name only a few. Alternatively, the phospholipid (phosphosphingolipid or a glycerophospholipid) may comprise cholesterol as membrane anchoring domain. Cholesterol-based membrane anchoring domains are, for instance, described in Achalkumar et al, “Cholesterol-based anchors and tethers for phospholipid bilayers and for model biological membranes”, Soft Matter, 2010, 6, 6036-6051. In illustrative embodiments the linker moiety of such a membrane anchoring domain comprises 1 to about 550 main chain atoms, 1 to about 500 main chain atoms, 1 to about 450 main chain atoms, 1 to about 350 main chain atoms, 1 to about 300 main chain atoms, 1 to about 250 main chain atoms, 1 to about 200 main chain atoms, 1 to about 150 main chain atoms, 1 to about 100 main chain atoms, 1 to about 50 main chain atoms, 1 to about 30 main chain atoms, 1 to about 20 main chain atoms, 1 to about 15 main chain atoms, or 1 to about 12 main chain atoms, or 1 to about 10 main chain atoms, wherein the main chain atoms are carbon atoms that are optionally replaced by one or more heteroatoms selected from the group consisting of N, O, P and S.

In some further aspects, the present invention relates to polymersomes capable of eliciting a CD8(+) T cell and/or CD4(+) T cell-mediated immune response.

In some aspects, the present invention relates to polymersomes capable of targeting of lymph node-resident macrophages and/or B cells. Exemplary non-limiting targeting mechanisms envisaged by the present invention include: i) delivery of conjugated antigens (e.g., polypeptides, etc.) to dendritic cells (DCs) for T cell activation (CD4 and/or CD8). Another one is: ii) delivery of whole folded antigens (e.g., proteins, etc.) that will be route to DC and will also trigger a titer (B cells).

In some aspects, the present invention relates to polymersomes with a conjugated antigen selected from a group consisting of: i) a self-antigen, ii) a non-self antigen, iii) a non-self immunogen and iv) a self-immunogen. Accordingly, the products and methods of the present invention are suitable for uses in settings (e.g., clinical settings) of induced tolerance, e.g., when targeting an autoimmune disease.

In some aspects, the present invention relates to polymersomes of the present invention comprising a lipid polymer.

The polymersomes of the present invention can also have co-encapsulated (i.e. encapsulated in addition to the antigen) one or more adjuvants. Examples of adjuvants include synthetic oligodeoxynucleotides (ODNs) containing unmethylated CpG motifs which can trigger cells that express Toll-like receptor 9 (including human plasmacytoid dendritic cells and B cells) to mount an innate immune response characterized by the production of Th1 and proinflammatory cytokines, cytokines such as Interleukin-1, Interleukin-2 or Interleukin-12, keyhole limpet hemocyanin (KLH), serum albumin, bovine thyroglobulin, or soybean trypsin inhibitor, too name only a few illustrative examples.

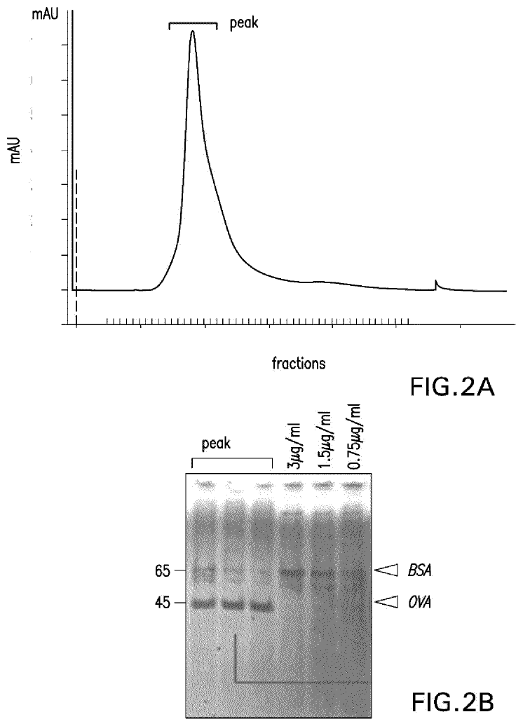

The polymersomes of the present invention can be of any size as long as the polymersomes are able to elicit an immune response. For example, the polymersomes may have a diameter of greater than 70 nm. The diameter of the polymersomes may range from about 100 nm to about 1 μm, or from about 100 nm to about 750 nm, or from about 100 nm to about 500 nm. The diameter of the polymersome may further range from about 125 nm to about 250 nm, from about 140 nm to about 240 nm, from about 150 nm to about 235 nm, from about 170 nm to about 230 nm, or from about 220 nm to about 180 nm, or from about 190 nm to about 210 nm. The diameter of the polymersomes may, for example, about 200 nm; about 205 nm or about 210 nm. When used as a collection to elicit an immune response, the collection of polymersomes is typically a monodisperse population. The mean diameter of the used collection/population of polymersomes is typically above 70 nm or above 125 nm, or above 140 nm, or above 150 nm, or above 160 nm, or for above 170 nm, or above 180 nm, or above 190 nm. The mean diameter of the collection of polymersomes may, for example, also in range of the individual polymersomes mentioned above, meaning the mean diameter of the collection of polymersomes may be in the range of 100 nm to about 1 μm, or in the range of about 100 nm to about 750 nm, or in the range of about 100 nm to about 500 nm, or in the range from about 125 nm to about 250 nm, from about 140 nm to about 240 nm, from about 150 nm to about 235 nm, from about 170 nm to about 230 nm, or from about 220 nm to about 180 nm, or from about 190 nm to about 210 nm. The mean diameter of the collection of polymersomes may, for example, be about 200 nm; about 205 nm or about 210 nm (cf. also FIG. 2 in this respect). In this context it is noted that the diameter can, for example, be determined by a dynamic light scattering (DLS) instrument using Z-average (d, nm), a preferred DLS parameter. Z-average size is the intensity weighted harmonic mean particle diameter (cf. Example 1 and 2). In this context, it is noted that according to U.S. Pat. No. 8,323,696 of Hubbel et al, a collection of polymersomes should have a mean diameter of less than 70 nm to be able to elicit immune response. Similarly, Stano et al, supra, 2013, while wanting to use smaller polymersome, use due to technical constraints polymersomes having a diameter of 125 nm+/−15 nm to elicit an immune response. Thus, it is surprising that a population/collection of polymersomes of the present invention with a mean diameter of, for example, more than 150 nm are able to induce both a cellular and a humoral immune response (cf. Example section). Such a collection of polymersomes may be in a form suitable for eliciting an immune response, for example, by injection.

In some aspects, the present invention relates to compositions of the present invention suitable for intradermal, intraperitoneal, subcutaneous, intravenous, or intramuscular injection, or non-invasive administration of an antigen of the present invention. The composition may include a polymersome (e.g., carrier) of the present invention having a membrane (e.g., circumferential membrane) of an amphiphilic polymer. The composition further includes a soluble (e.g., solubilized) antigen conjugated to the membrane of the amphiphilic polymer of the polymersome. The compositions of the present invention may be used in antibody discovery, vaccine discovery, or targeted delivery.

In some aspects, polymersomes of the present invention have hydroxyl groups on their surface. In some further aspects, polymersomes of the present invention do not have hydroxyl groups on their surface.

In the present context, the term “encapsulated” means enclosed by a membrane (e.g., membrane of the polymersome of the present invention, e.g., embodied inside the lumen of said polymersome). With reference to an antigen the term “encapsulated” further means that said antigen is neither integrated into—nor covalently bound to—nor conjugated to said membrane (e.g., of a polymersome of the present invention). With reference to compartmentalization of the vesicular structure of polymersome as described herein the term “encapsulated” means that the inner vesicle is completely contained inside the outer vesicle and is surrounded by the vesicular membrane of the outer vesicle. The confined space surrounded by the vesicular membrane of the outer vesicle forms one compartment. The confined space surrounded by the vesicular membrane of the inner vesicle forms another compartment.

In the context of the present invention, the term “conjugated” means coupled or connected by a covalent bond and the term “exterior surface of the polymersome” means the outside surface of the polymersome vesicle.

In the present context, the term “antigen” means any substance that may be specifically bound by components of the immune system. Only antigens that are capable of eliciting (or evoking or inducing) an immune response are considered immunogenic and are called “immunogens”. Exemplary non-limiting antigens are polypeptides derived from a soluble portion of proteins, hydrophobic polypeptides rendered soluble for conjugation and/or encapsulation as well as aggregated polypeptides that are soluble as aggregates. The antigen may originate from within the body (“self-antigen”) including a neoantigen (the term “neoantigen is used in its standard meaning to refer to an antigen that is as such absent from the normal (human) genome but is generated by mutagenesis within the body and compared with nonmutated self-antigens, is of relevance to tumor control), or from the external environment (“non-self”).

Membrane proteins form a class of antigens that typically produce a low immune response level. Of specific interest, soluble (e.g., solubilized) membrane proteins (MPs) and membrane-associated peptides (MAPs) and fragments (i.e., portions) thereof (e.g., the antigens mentioned herein) are conjugated to a polymersome, which may allow them to present to the immune system in a physiologically relevant manner to elicit immune response. This greatly boosts the immunogenicity of such antigens so that when compared to free antigens, a smaller amount of the corresponding antigen can be used to produce the same level of the immune response. Furthermore, the larger size of the polymersomes (compared to free membrane proteins) allows them to be detected by the immune system more easily.

In the present context, the term “Influenza hemagglutinin (HA)” refers to a glycoprotein found on the surface of influenza viruses. HA has at least 18 different antigens, which are all within the scope of the present invention. These subtypes are named H1 through H18. Non-limiting examples of “Influenza hemagglutinin (HA)” subtype H1 include SEQ ID NOs: 2, 3, 4 and 5.

In the present context, the term “Swine Influenza hemagglutinin (HA)” refers to a glycoprotein found on the surface of swine influenza viruses, which is a family of influenza viruses endemic in pigs. Non-limiting example of “Swine Influenza hemagglutinin (HA)” include subtype H1 SEQ ID NO: 3.

In the present context, the term “oxidation-stable” refers to a measure of polymersomes (or the corresponding polymers or membranes) resistance to oxidation, for example, using the method described by Scott et al., 2012, In this method a polymersome with an encapsulated antigen is incubated in a 0.5% solution of hydrogen peroxide and the amount of free (released) antigen can be quantified with UV/fluorescence HPLC. Polymersomes which release a substantial or all of the encapsulated antigen under these oxidizing conditions are considered to be oxidation sensitive. Another method of determining whether a block-copolymer and thus the resulting polymersome is oxidation stable or oxidation-sensitive is described in column 16 of U.S. Pat. No. 8,323,696. According to this method, polymers with functional groups that are oxidation-sensitive will be chemically altered by mild oxidizing agents, with a test for the same being enhanced solubility to 10% hydrogen peroxide for 20 h in vitro. As, for example, poly(propylene sulfide) (PPS) is an oxidation-sensitive polymer (see, for example, Scott et al 2012, supra and U.S. Pat. No. 8,323,696) PPS can serve as a reference to determine whether a polymer of interest and the respective polymersome of interest is oxidation-sensitive or oxidation stable, If, for example, the same or a higher amount of antigen, or about 90% or more of the amount, or about 80% or more, or about 70% or more, or about 60% or more is released from polymersomes of interest as it is from a PPS polymersome that has encapsulated therein the same antigen, then the polymersome is considered oxidation sensitive. If about only 0.5% or less, or about only 1.0% or less, or about 2% or less, or about 5% of less, or about 10% or less, or about 20% or less, or about 30% or less, or about 40% or less or about 50% or less of antigen is released from polymersomes of interest as it is from a PPS polymersome that has encapsulated therein the same antigen, then the polymersome is considered oxidation-stable. Thus, in line with this, PPS polymersomes as described in U.S. Pat. No. 8,323,696 or. PPS-bl-PEG polymersomes, e.g., made from poly(propylene sulfide) (PPS) and poly(ethylene glycol) (PEG) as components as described in Stano et al, are not oxidation-stable polymersomes within the meaning of the present invention. Similarly, PPS30-PEG17 polymersomes are not oxidation-stable polymersomes within the meaning of the present invention. Other non-limiting examples of measuring oxidation stability include measurement of stability in the presence of serum components (e.g., mammalian serum, e.g., human serum components) or stability inside an endosome, for example.

In the present context, the term “reduction-stable” refers to a measure of polymersome resistance to reduction in a reducing environment.

In the present context, the term “serum” refers to blood plasma from which the clotting proteins have been removed.

In the present context, the term “oxidation-independent release” refers to a release of the polymersome content without or essentially without oxidation of the polymers forming the polymersomes.

The term “polypeptide” is equally used herein with the term “protein”. Proteins (including fragments thereof, preferably biologically active fragments, and peptides, usually having less than 30 amino acids, e.g., up to 10 or more amino acids, e.g., 4, 5, 6, 7, 8, 9, 10, 11, 12, 13, 14, 15, 16, 17, 18, 19, 20, 25 or 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected e.g. by post-translational modifications like glycosylation, acetylation, phosphorylation and the like. Such modifications are well known in the art.

In the present context, the term “carbohydrates” refers to compounds such as aldoses and ketoses having the stoichiometric formula Cₙ(H₂O)ₙ (e.g., hence “hydrates of carbon”). The generic term “carbohydrate” includes, but is not limited to, monosaccharides, oligosaccharides and polysaccharides as well as substances derived from monosaccharides by reduction of the carbonyl group (alditols), by oxidation of one or more terminal groups to carboxylic acids, or by replacement of one or more hydroxy group(s) by a hydrogen atom, an amino group, thiol group or similar groups. It also includes derivatives of these compounds.

In the present context, the term “polynucleotide” (also “nucleic acid”, which can be used interchangeably with the term “polynucleotide”) refers to macromolecules made up of nucleotide units which e.g., can be hydrolysable into certain pyrimidine or purine bases (usually adenine, cytosine, guanine, thymine, uracil), d-ribose or 2-deoxy-d-ribose and phosphoric acid. Non-limiting examples of “polynucleotide” include DNA molecules (e.g. cDNA or genomic DNA), any backbone of oligonucleotides (e.g., phosphorothioate, 2′-O methyl, 2′ fluoro etc.) and their derivatives (e.g., lipid/cholesterol/polysaccharide modified oligos), RNA (mRNA), combinations thereof or hybrid molecules comprised of DNA and RNA. The nucleic acids can be double- or single-stranded and may contain double- and single-stranded fragments at the same time. Most preferred are double stranded DNA molecules and mRNA molecules.

In the present context, the term “antisense oligonucleotide” refers to a nucleic acid polymer, at least a portion of which is complementary to a nucleic acid which is present in a normal cell or in an affected cell. Exemplary “antisense oligonucleotide” include antisense RNA, siRNA, RNAi.

In the present context, the term “CD8(+) T cell-mediated immune response” refers to the immune response mediated by cytotoxic T cells (also known as TC, cytotoxic T lymphocyte, CTL, T-killer cells, cytolytic T cells, CD8(+) T-cells or killer T cells). Example of cytotoxic T cells include, but are not limited to antigen-specific effector CD8(+) T cells. In order for the T-cell receptors (TCR) to bind to the class I MHC molecule, the former must be accompanied by a glycoprotein called CD8, which binds to the constant portion of the class I MHC molecule. Therefore, these T cells are called CD8(+) T cells. Once activated, the TC cell undergoes “clonal expansion” with the help of the cytokine Interleukin-2 (IL-2), which is a growth and differentiation factor for T cells. This increases the number of cells specific for the target antigen that can then travel throughout the body in search of antigen-positive somatic cells.

In the present context, the term “clonal expansion of antigen-specific CD8(+) T cells” refers to an increase in the number of CD8(+) T cells specific for the target antigen.

In the present context, the term “cellular immune response” refers to an immune response that does not involve antibodies, but rather involves the activation of phagocytes, antigen-specific cytotoxic T-Iymphocytes, and the release of various cytokines in response to an antigen.

In the present context, the term “cytotoxic phenotype of antigen-specific CD8(+) T cells” refers to the set of observable characteristics of antigen-specific CD8(+) T cells related to their cytotoxic function.

In the present context, the term “lymph node-resident macrophages” refers to macrophages, which are large white blood cell that is an integral part of our immune system that use the process of phagocytosis to engulf particles and then digest them, present in lymph nodes that are small, bean-shaped glands throughout the body.

In the present context, the term “humoral immune response” refers to an immune response mediated by macromolecules found in extracellular fluids such as secreted antibodies, complement proteins, and certain antimicrobial peptides. Its aspects involving antibodies are often called antibody-mediated immunity.

In the present context, the term “B cells”, also known as B lymphocytes, are a type of white blood cell of the lymphocyte subtype. They function in the humoral immunity component of the adaptive immune system by secreting antibodies.

An “antibody” when used herein is a protein comprising one or more polypeptides (comprising one or more binding domains, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2, with IgG being preferred in the context of the present invention. An antibody relating to the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the IgG4 subtype would not have the capability to exert ADCC.

The term “antibody” also includes, but is not limited to, but encompasses monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

The antibody relating to the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

The term “essentially non-immunogenic” means that the block copolymer or amphiphilic polymer of the present invention does not elicit an adaptive immune response, i.e., in comparison to a conjugated immunogen, the block copolymer or amphiphilic polymer shows an immune response of less than 30%, preferably 20%, more preferably 10%, particularly preferably less than 9, 8, 7, 6 or 5%.

The term “essentially non-antigenic” means that the block copolymer or amphiphilic polymer of the present invention does not bind specifically with a group of certain products that have adaptive immunity (e.g., T cell receptors or antibodies), i.e., in comparison to a conjugated antigen the block copolymer or amphiphilic polymer shows binding of less than 30%, preferably 20%, more preferably 10%, particularly preferably less than 9, 8, 7, 6 or 5%.

Typically, binding is considered specific when the binding affinity is higher than 10⁻⁶M. Preferably, binding is considered specific when binding affinity is about 10⁻¹¹ to 10⁻⁸ M (KD), preferably of about 10⁻¹¹ to 10⁻⁶ M. If necessary, nonspecific binding can be reduced without substantially affecting specific binding by varying the binding conditions.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

“Polyclonal antibodies” or “polyclonal antisera” refer to immune serum containing a mixture of antibodies specific for one (monovalent or specific antisera) or more (polyvalent antisera) antigens which may be prepared from the blood of animals immunized with the antigen or antigens.

Furthermore, the term “antibody” as employed in the invention also relates to derivatives or variants of the antibodies described herein which display the same specificity as the described antibodies. Examples of “antibody variants” include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function (s) (see, e.g., U.S. Pat. No. 5,648,260).

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any method. For example, the monoclonal antibodies to be used in accordance with the present invention may be made by the hybridoma method first described by Kohler et al., Nature, 256: 495 (1975), or may be made by recombinant DNA methods (see, e.g., U.S. Pat. No. 4,816, 567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain (s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences.

“Humanized” forms of non-human (e.g., murine) antibodies are chimeric immunoglobulins, immunoglobulin chains or fragments thereof (such as Fv, Fab, Fab′, F (ab′) 2 or other antigen-binding subsequences of antibodies) of mostly human sequences, which contain minimal sequence derived from non-human immunoglobulin. For the most part, humanized antibodies are human immunoglobulins (recipient antibody) in which residues from a hypervariable region (also CDR) of the recipient are replaced by residues from a hypervariable region of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody optimally also will comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

The term “human antibody” includes antibodies having variable and constant regions corresponding substantially to human germline immunoglobulin sequences known in the art, including, for example, those described by Kabat et al. (See Kabat, et al. (1991) loc. cit.). The human antibodies of the invention may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence.

As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term thus preferably excludes sequences generated by genomic rearrangement in an immune cell.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). In one embodiment, the bispecific antibody comprises a first binding domain polypeptide, such as a Fab′ fragment, linked via an immunoglobulin constant region to a second binding domain polypeptide.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green etal. (1994) Nature Genetics 7:13-21, US 2003-0070185, WO 96/34096, and WO96/33735.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 171496; EP 173494, GB 2177096. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor etal, Immunology Today, 4: 7279, 1983; Olsson et al., Meth. Enzymol., 92: 3-16, 1982), and may be made according to the teachings of WO 92/06193 or EP 239400).

An antibody or fragment thereof may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et at. (1992) J. Mol. Biol. 227:776-798; Cook, G. P. et al. (1995) Immunol. Today Vol. 16 (5): 237-242; Chothia, et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

“Effector cells”, preferably human effector cells are leukocytes which express one or more FcRs and perform effector functions. Preferably, the cells express at least FcyRm and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood.

Techniques for production of antibodies, including polyclonal, monoclonal, humanized, bispecific and heteroconjugate antibodies are known in the art, some of which are exemplified below.

1) Polyclonal Antibodies.

Polyclonal antibodies are generally raised in animals by multiple subcutaneous (sc) or intraperitoneal (ip) injections of the relevant antigen (e.g., conjugated to a polymersome) and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable used to enhance the immune response.

The term “immunizing” refers to the step or steps of administering one or more antigens to a non-human animal so that antibodies can be raised in the animal.

Specifically, the non-human animal is preferably immunized at least two, more preferably three times with said polypeptide (antigen), optionally in admixture with an adjuvant. An “adjuvant” is a nonspecific stimulant of the immune response. The adjuvant may be in the form of a composition comprising either or both of the following components: (a) a substance designed to form a deposit protecting the antigen (s) from rapid catabolism (e.g. mineral oil, alum, aluminium hydroxide, liposome or surfactant (e.g. pluronic polyol) and (b) a substance that nonspecifically stimulates the immune response of the immunized host animal (e.g. by increasing lymphokine levels therein).

Exemplary molecules for increasing lymphokine levels include lipopolysaccaride (LPS) or a Lipid A portion thereof; Bordetalla pertussis; pertussis toxin; Mycobacterium tuberculosis; and muramyl dipeptide (MDP). Examples of adjuvants include Freund's adjuvant (optionally comprising killed M. tuberculosis; complete Freund's adjuvant); aluminium hydroxide adjuvant; and monophosphoryl Lipid A-synthetic trehalose dicorynomylcolate (MPL-TDM).

The “non-human animal” to be immunized herein may be a rodent. A “rodent” is an animal belonging to the Rodentia order of placental mammals. Exemplary rodents include mice, rats, guinea pigs, squirrels, hamsters, ferrets etc, with mice being the preferred rodent for immunizing according to the method herein. Other non-human animals which can be immunized herein include non-human primates such as Old World monkeys (e.g. baboons or macaques, including Rhesus monkeys and cynomolgus monkeys; see U.S. Pat. 5,658,570); but also non-mammals such as birds (e.g. chicken, or turkey); fish (for example, fish cultivated in aquaculture such as salmon, trout, or tilapia) or crustacean (such as shrimps or prawns) or other mammalian (life stock) animals such as rabbits; goats; sheep; cows; horses; pigs; donkeys or cats or dogs, for example.

By “screening” is meant subjecting one or more monoclonal antibodies (e.g., purified antibody and/or hybridoma culture supernatant comprising the antibody) to one or more assays which determine qualitatively and/or quantitatively the ability of an antibody to bind to an antigen of interest.

By “immuno-assay” is meant an assay that determines binding of an antibody to an antigen, wherein either the antibody or antigen, or both, are optionally adsorbed on a solid phase (i. e., an “immunoadsorbent” assay) at some stage of the assay. Exemplary such assays include ELISAs, radioimmunoassays (RIAs), and FACS assays. Given the above, the present invention provides thus a monoclonal or polyclonal antibody obtainable by the aforedescribed methods for the generation of an antibody, i.e., by immunizing a non-human animal as described before.

2) Monoclonal Antibodies.

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256: 495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization.

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cell, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassus, Virginia USA. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed again desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107: 220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5: 256-262 (1993) and Plückthun, Immunol. Revs. 130: 151-188 (1992).

3) Humanized Antibodies

The antibodies of the invention may further comprise humanized or human antibodies. Humanized forms of non-human (e.g., murine) antibodies are chimeric immunoglobulins, immunoglobulin chains or fragments thereof (such as Fv, Fab, Fab′, F (ab′) 2 or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues.

Humanized antibodies may also comprise residues which are found neither in the recipient antibody nor in the imported CDR or framework sequences. In general, the humanized antibody will comprise substantially all of at least one, and typically two, variable domain, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321: 522-525 (1986); Riechmann et al., Nature 332: 323-327 (1988); Verhoeyen et al., Science 239: 1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol.) 151: 2296 (1993); Chothia et al., J. Mol. Biol., 196: 901 (1987).

Another method uses a particular framework derived from the consensus sequence of all human antibodies of a particular subgroup of light or heavy chains. The same framework may be used for several different humanized antibodies. Carter et al., Proc. Natl. Acad. Sci. USA, 89: 4285 (1992); Presta et al., J. Immunol., 151: 2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen (s), is achieved. In general, the CDR residues are directly and most substantially involved in influencing antigen binding.

Various forms of the humanized antibody are contemplated. For example, the humanized antibody may be an antibody fragment, such as a Fab, which is optionally conjugated with one or more cytotoxic agent (s) in order to generate an immunoconjugate.

Alternatively, the humanized antibody may be an intact antibody, such as an intact IgGI antibody.

4) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255-258 (1993); Bruggermann et al., Year in Immun., 7: 33 (1993); U.S. Pat. Nos. 5,591, 669 and WO 97/17852.

Alternatively, phage display technology can be used to produce human antibodies and antibody fragments in vitro, from immunoglobulin variable (V) domain gene repertoires from unimmunized donors. McCafferty et al., Nature 348: 552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3: 564-571 (1993). Several sources of V-gene segments can be used for phage display. Clackson et al., Nature 352: 624-628 (1991) isolated a diverse array of anti-oxazolone antibodies from a small random combinatorial library of V genes derived from the spleens of immunized mice. A repertoire of V genes from unimmunized hman donors can be constructed and antibodies to a diverse array of antigens (including self-antigens) can be isolated essentially following the techniques described by Marks et al., J. Mol. Biol. 222: 581-597 (1991), or Griffith et al., EMBO J. 12: 725-734 (1993). See also, U.S. Pat. Nos. 5,565,332 and 5,573,905.

The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol. 147 (1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resemble that seen in human in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995). Finally, human antibodies may also be generated in vitro by activated B cells (see U.S. Pat. Nos 5,567,610 and 5,229,275).

5) Bispecific and Polyspecific Antibodies

Bispecific antibodies (BsAbs) are antibodies that have binding specificities for at least two different epitopes, including those on the same or another protein. Alternatively, one arm can be armed to bind to the target antigen, and another arm can be combined with an arm that binds to a triggering molecule on a leukocyte such as a T-cell receptor molecule (e.g., CD3), or Fc receptors for IgG (FcyR) such as FcyRI (CD64), FcyRII (CD32) and FcyRin (CD16), so as to focus and localize cellular defense mechanisms to the target antigen-expressing cell. Such antibodies can be derived from full length antibodies or antibody fragments (e.g. F(ab′)₂ bispecific antibodies).

Bispecific antibodies may also be used to localize cytotoxic agents to cells which express the target antigen. Such antibodies possess one arm that binds the desired antigen and another arm that binds the cytotoxic agent (e.g., methotrexate).

Methods for making bispecific antibodies are known in the art. Traditional production of full length bispecific antibodies is based on the coexpression of two immunoglobulin heavy chain-light chain pairs, where the two chains have different specificities. Millstein et al., Nature, 305: 537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10: 3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains (s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F (ab′) 2 molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148 (5): 1547-1553 (1992).

The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. USA, 90: 6444-6448 (1993) has provided an alternative mechanism for making bispecific/bivalent antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific/bivalent antibody fragments by the use of single-chain Fv (sFv) dimers has also been reported. See Gruber et al., J. Imnzunol., 152: 5368 (1994).

Antibodies with more than two valencies are contemplated. For example, trispecific antibodies can be prepared. Tutt et al., J. Immunol. 147: 60 (1991).

Exemplary bispecific antibodies may bind to two different epitopes on a given molecule. Alternatively, an anti-protein arm may be combined with an arm which binds to a triggering molecule on a leukocyte such as a T-cell receptor molecule (e.g., CD2, CD3, CD28 or B7), or Fc receptors for IgG (FcyR), such as FcyRI (CD64), FcyRII (CD32) and FcyRIII (CD16) so as to focus cellular defense mechanisms to the cell expressing the particular protein.

Another bispecific antibody of interest binds the protein of interest and further binds Human Serum Albumin.

The “diabody” technology described by Hollinger et al. , Proc. Natl. Acad. Sci. USA, 90: 6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152: 5368 (1994).

Antibodies with more than two valencies are contemplated. For example, trispecific antibodies can be prepared. Tutt et al. J. Immunol. 147: 60 (1991).

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain (s) comprise two or more variable domains. For instance, the polypeptide chain (s) may comprise VDI (X1ₙ-VD2-(X2)n-Fc, wherein VDI is a first variable domain, VD2 is a second variable domain, Fc is one polypeptide chain of an Fc region, X1 and X2 represent an amino acid or polypeptide, and n is 0 or 1. For instance, the polypeptide chain (s) may comprise: VH-CHI-flexible linker-VH-CHI-Fc region chain; or VH-CHI-VH-CHI-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

6) Heteroconjugate Antibodies

Heteroconjugate antibodies are also within the scope of the present invention.

Heteroconjugate antibodies are composed of two covalently joined antibodies. For example, one of the antibodies in the heteroconjugate can be coupled to avidin, the other to biotin. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

For additional antibody production techniques, see “Antibodies: A Laboratory Manual”, eds. Harlow et al., Cold Spring Harbor Laboratory, 1988. The present invention is not necessarily limited to any particular source, method of production, or other special characteristics of an antibody.

The antibody relating to the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

Non-limiting examples of cancers include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation, lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary, plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 or PD-L1 antibody), and recurrent cancers.

The term “subject” is intended to include living organisms. Examples of subjects include mammals, e.g., humans, dogs, cows, horses, pigs, sheep, goats, cats, mice, rabbits, rats, and transgenic non-human animals. In preferred embodiments of the invention, the subject is a human.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the subject's own immune system. The term “patient” includes human and other mammalian subjects that receive either prophylactic or therapeutic treatment.

The appropriate dosage, or therapeutically effective amount, of the antibody or antigen binding portion thereof will depend on the condition to be treated, the severity of the condition, prior therapy, and the patient's clinical history and response to the therapeutic agent. The proper dose can be adjusted according to the judgment of the attending physician such that it can be administered to the patient one time or over a series of administrations. The pharmaceutical composition can be administered as a sole therapeutic or in combination with additional therapies as needed.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

Pharmaceutical compositions for injection may be presented in unit dosage form, e.g., in ampoules or in multi-dose containers, with an added preservative. In addition, a number of recent drug delivery approaches have been developed and the pharmaceutical compositions of the present invention are suitable for administration using these new methods, e. g., Inject-ease, Genject, injector pens such as Genen, and needleless devices such as MediJector and BioJector. The present pharmaceutical composition can also be adapted for yet to be discovered administration methods. See also Langer, 1990, Science, 249: 1527-1533.

The pharmaceutical composition can also be formulated as a depot preparation. Such long acting formulations may be administered by implantation (for example subcutaneously, into the ligament or tendon, subsynovially or intramuscularly), by subsynovial injection or by intramuscular injection. Thus, for example, the formulations may be modified with suitable polymeric or hydrophobic materials (for example as a emulsion in an acceptable oil) or ion exchange resins, or as sparingly soluble derivatives, for example, as a sparingly soluble salt.

The pharmaceutical compositions may also be in a variety of conventional depot forms employed for administration to provide reactive compositions. These include, for example, solid, semi-solid and liquid dosage forms, such as liquid solutions or suspensions, slurries, gels, creams, balms, emulsions, lotions, powders, sprays, foams, pastes, ointments, salves, balms and drops.

The pharmaceutical compositions may, if desired, be presented in a vial, pack or dispenser device which may contain one or more unit dosage forms containing the active ingredient. In one embodiment, the dispenser device can comprise a syringe having a single dose of the liquid formulation ready for injection. The syringe can be accompanied by instructions for administration.

The pharmaceutical composition may further comprise additional pharmaceutically acceptable components. Other pharmaceutically acceptable carriers, excipients, or stabilizers, such as those described in Remington's Pharmaceutical Sciences 16th edition, Osol, A. Ed. (1980) may also be included in a protein formulation described herein, provided that they do not adversely affect the desired characteristics of the formulation. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, asparagine, 2-phenylalanine, and threonine; sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

The formulations described herein are useful as pharmaceutical compositions in the treatment and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

As used herein, the term “treating” and “treatment” refers to administering to a subject a therapeutically effective amount of a pharmaceutical composition according to the invention. A “therapeutically effective amount” refers to an amount of the pharmaceutical composition or the antibody which is sufficient to treat or ameliorate a disease or disorder, to delay the onset of a disease or to provide a therapeutic benefit in the treatment or management of a disease.

As used herein, the term “prophylaxis” refers to the use of an agent for the prevention of the onset of a disease or disorder. A “prophylactically effective amount” defines an amount of the active component or pharmaceutical agent sufficient to prevent the onset or recurrence of a disease.

As used herein, the terms “disorder” and “disease” are used interchangeably to refer to a condition in a subject. In particular, the term “cancer” is used interchangeably with the term “tumor”.

The kit of the invention will typically comprise the container described above and one or more other containers comprising materials desirable from a commercial and user standpoint, including buffers, diluents, filters, needles, syringes, and package inserts with instructions for use.

In the present context, the term “liposome” refers to a spherical vesicle having at least one lipid bilayer.

In the present context, the term “endosome” refers to a membrane-bound compartment (i.e., a vacuole) inside eukaryotic cells to which materials ingested by endocytosis are delivered.

In the present context, the term “late-endosome” refers to a pre-lysosomal endocytic organelle differentiated from early endosomes by lower lumenal pH and different protein composition. Late endosomes are more spherical than early endosomes and are mostly juxtanuclear, being concentrated near the microtubule organizing center.

In the present context, the term “T helper cells” (also called TH cells or “effector CD4(+) T cells”) refers to T lymphocytes that assist other white blood cells in immunologic processes, including maturation of B cells into plasma cells and memory B cells, and activation of cytotoxic T cells and macrophages. These cells are also known as “CD4(+) T cells” because they express the CD4 glycoprotein on their surfaces. Helper T cells become activated when they are presented with e.g., peptide antigens, by MHC class II molecules, which are expressed on the surface of antigen-presenting cells (APCs).

As used herein, the term “self-antigen” refers to any molecule or chemical group of an organism which acts as an antigen in inducing antibody formation in another organism but to which the healthy immune system of the parent organism is tolerant.

As used herein, the term “% identity” refers to the percentage of identical amino acid residues at the corresponding position within the sequence when comparing two amino acid sequences with an optimal sequence alignment as exemplified by the ClustalW or X techniques as available from www.clustal.org, or equivalent techniques. Accordingly, both sequences (reference sequence and sequence of interest) are aligned, identical amino acid residues between both sequences are identified and the total number of identical amino acids is divided by the total number of amino acids (amino acid length). The result of this division is a percent value, i.e. percent identity value/degree.

Immunization method of the present invention can be carried out using a full-sized soluble conjugated antigen (e.g., protein (instead of fragment thereof) in a synthetic environment that allows its proper folding, and therefore the probability of isolating antibodies capable of detecting corresponding antigens (e.g., a membrane protein) in vivo would be higher. Moreover, the immunization and antibody generation can be carried out without any prior knowledge of the membrane protein structure, which may otherwise be necessary when using a peptide-based immunization approach.

Further, when compared to other techniques, the method of the present invention allows for a rapid and cost-effective production of membrane protein conjugated to an oxidation-stable membrane.

In some aspects, the present invention relates to a method for eliciting an immune response to an antigen (e.g., an immunogen) in a subject. The method may include injecting the subject with a composition including a polymersome of the present invention having a membrane (e.g., circumferential) of an amphiphilic polymer. The composition further includes a soluble antigen conjugated to the membrane of the amphiphilic polymer of the polymersome of the present invention. The immunogen may be a membrane-associated protein. In some further aspects, the polymersome of the present invention comprises a lipid polymer.

The frequency of the injection may be determined and adjusted by a person skilled in the art, dependent on the level of response desired. For example, weekly or bi-weekly injections of polymersomes of the present invention may be given to the subject, which may include a mammalian animal. The immune response can be measured by quantifying the blood concentration level of antibodies in the mammalian animal against the initial amount of antigen conjugated to the polymersome of the present invention.

The structure of the polymersomes may include amphiphilic block copolymers self-assembled into a vesicular format and conjugating various antigens (e.g., soluble proteins, etc.), that are conjugated by methods described herein (e.g., Examples 1 and 2 as described herein).

In the present context, the term “soluble antigen” as used herein means an antigen capable of being dissolved or liquefied. The term “soluble antigen” also includes antigens that were “solubilized”, i.e., rendered soluble or more soluble, especially in water, by the action of a detergent or other agent. Exemplary non-limiting soluble antigens of the present invention include: polypeptides derived from a non-soluble portion of proteins, hydrophobic polypeptides rendered soluble for conjugation as well as aggregated polypeptides that are soluble as aggregates.

In some aspects, the antigens (e.g., membrane proteins) of the present invention are solubilized with the aid of detergents, surfactants, temperature change or pH change. The vesicular structure provided by the amphiphilic block copolymers allows the antigens (e.g., membrane protein) to be folded in a physiologically correct and functional manner, allowing the immune system of the target mammalian animal to detect said antigens, thereby producing a strong immune response.

In some aspects, the injection of the composition of the present invention may include intraperitoneal, subcutaneous, or intravenous, intramuscular injection, or non-invasive administration. In some other aspects, the injection of the composition of the present invention may include intradermal injection.

In some other aspects, the immune response level may be further heightened or boosted by including an adjuvant in the composition including the polymersome of the present invention. In such aspects, the polymersome and the adjuvant can be administered simultaneously to the subject.

In some aspects, a block copolymer or an amphiphilic polymer of the polymersome of the present invention is neither immunostimulant nor adjuvant.

In some other aspects, a block copolymer or an amphiphilic polymer of the polymersome of the present invention is immunostimulant and/or adjuvant.

In some further aspects, a polymersome of the present invention is immunogenic.

In some further aspects, a polymersome of the present invention is non-immunogenic.

In some aspects, the adjuvant may be administered separately from the administration of the composition of the present invention including the polymersome of the present invention. The adjuvant may be administered before, simultaneously, or after the administration of the composition including the polymersome conjugated to an antigen of the present invention. For example, the adjuvant may be injected to the subject after injecting the composition including the polymersome conjugated to an antigen of the present invention. In some aspects, the adjuvant can be encapsulated together with the antigen conjugated to the polymersomes.

A person skilled in the art would readily recognize and appreciate that the types of adjuvant to be injected or also orally administered, for example, depend on the types of antigen to be injected. The antigen may be an antigen of bacterial, viral, or fungi origin. For example, in the case where the antigen is OVA, the adjuvant may be Sigma Adjuvant System (SAS). Other antigen-adjuvant pairs are also suitable for use in the methods of the present invention. In certain aspects, the use of adjuvants is not needed. In yet other aspects, the present method works better, i.e., stronger immune response being evoked, without the use of adjuvants.

In some aspects, a membrane protein may be a transmembrane protein, G protein-coupled receptor, neurotransmitter receptor, kinase, porin, ABC transporter, ion transporter, acetylcholine receptor and cell adhesion receptor. The membrane proteins may also be fused to or coupled with a tag or may be tag-free. If the membrane proteins are tagged, then the tag may, for example, be selected from well-known affinity tags such as VSV, His-tag, Strep-tag®, Flag-tag, Intein-tag or GST-tag or a partner of a high affinity binding pair such as biotin or avidin or from a label such as a fluorescent label, an enzyme label, NMR label or isotope label.

In some aspects, the membrane proteins of fragments (or portions) thereof may be presented prior to conjugation, or conjugated simultaneously with the production of the protein through a cell-free expression system. The cell-free expression system may be an in vitro transcription and translation system.

The cell-free expression system may also be an eukaryotic cell-free expression system such as the TNT system based on rabbit reticulocytes, wheat germ extract or insect extract, a prokaryotic cell-free expression system or an archaic cell-free expression system.

As mentioned above, the polymersomes may be formed of amphiphilic di-block or tri-block copolymers. In various aspects, the amphiphilic polymer may include at least one monomer unit of a carboxylic acid, an amide, an amine, an alkylene, a dialkylsiloxane, an ether or an alkylene sulphide.

In some aspects, the amphiphilic polymer used for the formation of a polymersome of the invention may be a polyether block selected from the group consisting of an oligo(oxyethylene) block, a poly(oxyethylene) block, an oligo(oxypropylene) block, a poly(oxypropylene) block, an oligo(oxybutylene) block and a poly(oxybutylene) block. Further examples of blocks that may be included in the polymer include, but are not limited to, poly(acrylic acid), poly(methyl acrylate), polystyrene, poly(butadiene), poly(2-methyloxazoline), poly(dimethyl siloxane), poly(e-caprolactone), poly(propylene sulphide), poly(N-isopropylacrylamide), poly(2-vinylpyridine), poly(2-(diethylamino)ethyl methacrylate), poly(2-diisopropylamino)ethylmethacrylate), poly(2-methacryloyloxy)ethylphosphorylcholine, poly (isoprene), poly (isobutylene), poly (ethylene-co-butylene) and poly(lactic acid). Examples of a suitable amphiphilic polymer include, but are not limited to, poly(ethyl ethylene)-b-poly(ethylene oxide) (PEE-b-PEO), poly(butadiene)-b-poly(ethylene oxide) (PBD-b-PEO), poly(styrene)-b-poly(acrylic acid) (PS-PAA), poly(2-methyloxazo1ine)-b-poly(dimethylsiloxane)-b-poly(2-methyloxazoline) (PMOXA-bPDMS-bPMOXA) including for example, triblock copolymers such as PMOXA₂₀-PDMS₅₄-PMOXA₂₀ (ABA) employed by May et al., 2013, poly(2-methyloxazoline)-b-poly(dimethylsiloxane)-b-poly(ethylene oxide) (PMOXA-b-PDMS-b-PEO), poly(ethylene oxide)-b-poly(propylene sulfide)-b-poly(ethylene oxide) (PEO-b-PPS-b-PEO) and a poly(ethylene oxide)-poly(butylene oxide) block copolymer. A block copolymer can be further specified by the average block length of the respective blocks included in a copolymer. Thus, PB_{M}PEO_{N} indicates the presence of polybutadiene blocks (PB) with a length of M and polyethylene oxide (PEO) blocks with a length of N. M and N are independently selected integers, which may for example be selected in the range from about 6 to about 60. Thus, PB₃₅PEO₁₈ indicates the presence of polybutadiene blocks with an average length of 35 and of polyethylene oxide blocks with an average length of 18.

In certain aspects, the PB-PEO diblock copolymer comprises 5-50 blocks PB and 5-50 blocks PEO. Likewise, PB₁₀PEO₂₄ indicates the presence of polybutadiene blocks with an average length of 10 and of polyethylene oxide blocks with an average length of 24. Illustrative examples of suitable PB-PEO diblock copolymers that can be used in the present invention include the diblock copolymers PBD₂₁-PEO₁₄ (that is also commercially available) and [PBD]₂₁-[PEO]₁₂, (cf., WO2014/077781A1 and Nallani et al., 2011), As a further example E₀Bₚ indicates the presence of ethylene oxide blocks (E) with a length of 0 and butadiene blocks (B) with a length of P. Thus, O and P may be independently selected integers, e.g. in the range from about 10 to about 120. Thus, E₁₆E₂₂ indicates the presence of ethylene oxide blocks with an average length of 16 and of butadiene blocks with an average length of 22.

In certain aspects, the polymersome of the present invention may contain one or more compartments (or otherwise termed “multicompartments). Compartmentalization of the vesicular structure of polymersome allows for the co-existence of complex reaction pathways in living cell and helps to provide a spatial and temporal separation of many activities inside a cell. Accordingly, more than one type of antigens may be conjugated to the polymersome of the present invention. The different antigens may have the same or different isoforms. Each compartment may also be formed of a same or a different amphiphilic polymer. In various aspects, two or more different antigens are integrated into the circumferential membrane of the amphiphilic polymer. Each compartment may be conjugated to at least one of peptide, protein, and nucleic acid. The peptide, protein, polynucleotide or carbohydrate may be immunogenic.

Further details of suitable multicompartmentalized polymersomes can be found in WO 20121018306, the contents of which being hereby incorporated by reference in its entirety for all purposes.

The polymersomes may also be free-standing or immobilized on a surface, such as those described in WO 2010/1123462, the contents of which being hereby incorporated by reference in its entirety for all purposes.

In the case where the polymersome carrier contains more than one compartment, the compartments may comprise an outer block copolymer vesicle and at least one inner block copolymer vesicle, wherein the at least one inner block copolymer vesicle is encapsulated inside the outer block copolymer vesicle. In some aspects, each of the block copolymer of the outer vesicle and the inner vesicle includes a polyether block such as a poly(oxyethylene) block, a poly(oxypropylene) block, and a poly(oxybutylene) block. Further examples of blocks-that may be included in the copolymer include, but are not limited to, poly(acrylic acid), poly(methyl acrylate), polystyrene, poly(butadiene), poly(2-methyloxazoline), poly(dimethyl siloxane), poly(L-isocyanoalanine(2-thiophen-3-yl-ethyl)amide), poly(e-caprolactone), poly(propylene sulphide), poly(N-isopropylacrylamide), poly(2-vinylpyridine), poly(2-(diethylamino)ethyl methacrylate), poly(2-(diisopropylamino)ethylmethacrylate), poly(2-(methacryloyloxy)ethylphosphorylcholine) and poly(lactic acid). Examples of suitable outer vesicles and inner vesicles include, but are not limited to, poly(ethyl ethylene)-b-poly(ethylene oxide) (PEE-b-PEO), poly(butadiene)-b-poly(ethylene oxide) (PBD-b-PEO), poly(styrene)-b-poly(acrylic acid) (PS-b-PAA), poly(ethylene oxide)-poly(caprolactone) (PEO-b-PCL), poly(ethylene oxide)-poly(lactic acid) (PEO-b-PLA), poly(isoprene)-poly(ethylene oxide) (Pl-b-PEO), poly(2-vinylpyridine)-poly(ethylene oxide) (P2VP-b-PEO), poly(ethylene oxide)-poly(N-isopropylacrylamide) (PEO-b-PNIPAm), poly(ethylene glycol)-poly(propylene sulfide) (PEG-b-PPS), poly (methylphenylsilane)-poly(ethylene oxide) (PMPS-b-PEO-b-PMPS-b-PEO-b-PMPS), poly(2-methyloxazoline)-b-poly-(dimethylsiloxane)-b-poly(2-methyloxazoline) (PMOXA-b-PDMS-b-PMOXA), poly(2-methyloxazoline)-b-poly(dimethylsiloxane)-b-poly(ethyleneoxide) (PMOXA-b-PDMS-b-PEO), poly[styrene-b-poly(L-isocyanoalanine(2-thiophen-3-yl-ethyl)amide)] (PS-b-PIAT), poly(ethylene oxide)-b-poly(propylene sulfide)-b-poly(ethylene oxide) (PEO-b-PPS-b-PEO) and a poly(ethylene oxide)-poly(butylene oxide) (PEO-b-PBO) block copolymer. A block copolymer can be further specified by the average number of the respective blocks included in a copolymer. Thus PS_{M}-PIAT_{N} indicates the presence of polystyrene blocks (PS) with M repeating units and poly(L-isocyanoalanine(2-thiophen-3-yl-ethyl)amide) (PIAT) blocks with N repeating units. Thus, M and N are independently selected integers, which may for example be selected in the range from about 5 to about 95. Thus, PS₄₀-PIAT₅₀ indicates the presence of PS blocks with an average of 40 repeating units and of PIAT blocks with an average of 50 repeating units.

In some other aspects, the invention relates to polymersomes that in addition to the antigen being covalently coupled to the exterior surface also have an solubilized antigen encapsulated within (the interior of) the polymersome. The invention thus also relates to methods for production of an encapsulated antigen in polymersome including methods based on mixing a non-aqueous solution of polymers in aqueous solution of antigens, sonication of corresponding mixed solutions of polymers and antigens, or extrusion of corresponding mixed solutions of polymers and antigens. Exemplary methods include those described in Rameez et al, Langmuir 2009, and in Neil et al Langmuir 2009, 25(16), 9025-9029. In one embodiment of polymersomes that also have an antigen encapsulated therein, the encapsulation is carried out as described in co-pending European patent application 18153348.0, filed with the EPO on 25 Jan. 2018, the entire contents is incorporated herein by reference for all purposes.

Compared to existing uptake and cross-presentation vehicles and methods based thereon the polymersomes of present invention inter alia offer one or more of the following advantages that are also aspects of the present invention:
The polymersomes improve immunogenic properties of antigens conjugated to the exterior surface of said polymersomes via a covalent bond;
The polymersomes are very efficient in uptake and cross-presentation to the immune system;
The immune response comprises a CD8(+) T cell-mediated immune response;
The polymersomes are oxidation-stable;
Polymer bilayer of the polymersome is much more robust than a lipid bilayer;
The polymersome is not a micelle;
The polymersomes do not comprises oxidative-sensitive groups (e.g., under physiological conditions) to release an antigen from conjugation;
The humoral response is stronger compared to that produced by free antigen-based techniques (with or without adjuvants) and encapsulated antigens;
The immune response induced by polymersomes of the present invention could still be even further boosted using adjuvants;
The polymers of polymersomes of the present invention are inherently robust and can be tailored or functionalized to increase their circulation time in the body;
The polymersomes of the present invention are stable in the presence of serum components;
The polymers of polymersomes are inexpensive and quick to synthesize;
The amount of an antigen required to elicit an immune response by the methods of the present invention using polymersomes of the present invention is less compared to free antigen-based techniques (with or without adjuvants) and encapsulated antigens.

The invention is also characterized by the following items:
1. A polymersome capable of eliciting an immune response, comprising: an antigen selected from the group consisting of:
   a) a polypeptide (e.g., short peptides of up to 10 amino acids or longer peptides with more amino acids);
   b) a carbohydrate;
   c) a polynucleotide (e.g., DNA or RNA);
   d) a combination of (a) and/or (b) and/or (c);

wherein the antigen is conjugated to the exterior surface of the polymersome via a covalent bond.
2. The polymersome of item 1, wherein the covalent bond comprises: i) an amide moiety; and/or ii) a secondary amine moiety; and/or iii) a 1,2,3-triazole moiety (e.g., as described in van Dongen et al., 2008, supra), preferably said 1,2,3-triazole moiety is a 1,4-disubstituted[1,2,3]triazole moiety or a 1,5-disubstituted[1,2,3]triazole moiety (e.g., as described in Boren et al., 2008 supra); and/or iv) pyrazoline moiety (e.g., as described in de Hoog et al., 2012, supra); and/or v) thioether or disulfide moiety; and/or vi) ester moiety; and/or vii) carbamate and or carbonate moiety and/or viii) an ether moiety (bond).
3. The polymersome of item 2, wherein the covalent bond that conjugates the antigen to the exterior surface of the polymersome is formed by reacting a reactive group present on the exterior surface of the polymersome with a reactive group of the antigen (e.g., coupling via the carbonyl (CHO)-group generated by treatment of the polymersome with Dess-Martin periodinane and subsequent reduction of the formed carboxamide to a secondary amine).
4. The polymersome of item 3, wherein the covalent bond is selected from the group consisting of: i) a carboxamide bond; ii) a 1,4-disubstituted[1,2,3]triazole or 1,5-disubstituted[1,2,3]triazole bond; iii) a substituted pyrazoline bond; iv) thioether or disulfide bond; or v) secondary amine bond.
5. The polymersome of item 4, wherein: i) the reactive group present on the exterior surface of the polymersome is an aldehyde group and the reactive group of the antigen is an amine group, thereby forming the carboxamide group; or ii) the reactive group present on the exterior surface of the polymersome is an alkyne group and the reactive group of the antigen is an azide group, thereby forming the 1,2,3-triazole group, preferably via copper- or ruthenium catalyzed azide-alkyne cycloaddition, further preferably said 1,2,3-triazole is 1,4-disubstituted or 1,5-disubstituted; or iii) the reactive group present on the exterior surface of the polymersome is a methacrylate- and/or hydroxyl group and the reactive group of the antigen is a tetrazole group, thereby forming the pyrazoline group, preferably said forming of the pyrazoline group comprises a nitrile imine intermediate; iv) the reactive group present on the exterior surface of the polymersome is sulfhydryl-reactive chemical group (e.g., maleimide) and the reactive group of the antigen is a sulfhydryl group, thereby forming thioether bond or disulfide bond (e.g., via alkylation or disulfide exchange respectively); or v) the reactive group present on the exterior surface of the polymersome is aldehyde group and the reactive group of the antigen is an amine-containing group, thereby forming secondary amine bond (e.g., via a Schiff-base intermediate).
6. The polymersome of item 5, wherein the carboxamide bond has further been reacted with a reducing agent to form a secondary amine.
7. The polymersome of any of the foregoing items, wherein the covalent bond is formed via a linker moiety, wherein these linker molecules can be either aliphatic or aromatic.
8. The polymersome of item 7, wherein the linker moiety L is a peptidic linker or a straight or branched hydrocarbon-based linker.
9. The adapter molecule of item 7 or 8, wherein the linker moiety comprises 1 to about 550 main chain atoms (e.g., DSPE-PEG having 4000 with about 537 main chain atoms), 1 to about 500 main chain atoms, 1 to about 450 main chain atoms (e.g., DSPE-PEG3000 with about 408 main chain atoms), 1 to about 350 main chain atoms, 1 to about 300 main chain atoms (e.g., DSPE-PEG having 2000 with about 279 main chain atoms), 1 to about 250 main chain atoms, 1 to about 200 main chain atoms, 1 to about 150 main chain atoms, 1 to about 100 main chain atoms, 1 to about 50 main chain atoms, 1 to about 30 main chain atoms, 1 to about 20 main chain atoms, 1 to about 15 main chain atoms, or 1 to about 12 main chain atoms, or 1 to about 10 main chain atoms, wherein the main chain atoms are carbon atoms that are optionally replaced by one or more heteroatoms selected from the group consisting of N, O, P and S
10. The polymersome of any of items 7 to 9, wherein the linker moiety comprises a membrane anchoring domain which integrates the linker moiety into the membrane of the polymersome.
11. The polymersome of item 10, wherein the membrane anchoring domain comprise a lipid.
12. The polymersome of item 11, wherein the lipid is a phospholipid or a glycolipid.
13. The polymersome of item 12 wherein the glycolipid comprises glycophosphatidylinositol (GPI).
14. The polymersome of item 12, wherein the phospholipid is a phosphosphingolipid or a glycerophospholipid.
15. The polymersome of item 12, wherein the phosphosphingolipid comprises distearoylphosphatidylethanolamine [DSPE] conjugate to polyethylene glycol (PEG) (DSPE-PEG) or a cholesterol based conjugate.
16. The polymersome of item 15, wherein the DSPE-PEG comprises from 2 to about 500 ethylene oxide units (e.g., PEG2000 having 43 ethylene oxide units, so PEG20K having about 440 ethylene oxide units).
17. The polymersome of any of the foregoing items, wherein the linker is non-hydrolysable and/or non-oxidizable under physiological conditions.
18. The polymersome according to any one of preceding items, wherein the physiological conditions are characterized by: a temperature in the range of about 20-40° C., atmospheric pressure of 1 and pH in the range of about 6-8.
19. The polymersome according to any one of preceding items, wherein the polymersome has a vesicular morphology.
20. The polymersome according to any one of preceding items, wherein the elicited immune response comprises a humoral immune response and/or a cellular immune response.
21. The polymersome according to any one of preceding items, wherein the polymersome is an oxidation-stable polymersome.
22. The polymersome according to any one of preceding items, wherein the polymersome has a diameter greater than 70 nm, preferably said diameter ranging from about 100 nm to about 1 μm, or from about 100 nm to about 750 nm, or from about 100 nm to about 500 nm, or from about 125 nm to about 250 nm, from about 140 nm to about 240 nm, from about 150 nm to about 235 nm, from about 170 nm to about 230 nm, or from about 220 nm to about 180 nm, or from about 190 nm to about 210 nm.
23. The polymersome according to any one of preceding items, wherein the polymersome is oxidation-stable in the presence of serum components, preferably said oxidation-stability is an in vivo, ex vivo or in vitro oxidation-stability.
24. The polymersome according to any one of preceding items, wherein the polymersome is stable inside an endosome, preferably said stability is an in vivo, ex vivo or in vitro stability.
25. The polymersome according to any one of preceding items, wherein the polymersome has an improved oxidation stability compared to corresponding oxidation stability of a liposome or nanoparticle, preferably said improved stability is an in vivo, ex vivo and/or in vitro improved stability.
26. The polymersome according to any one of preceding items, wherein the polymersome is capable of releasing said antigen in an oxidation-independent manner and triggering a humoral immune response, wherein said releasing is an in vivo, ex vivo or in vitro releasing.
27. The polymersome according to any one of preceding items, wherein said humoral immune response comprises production of specific antibodies, further preferably said immune response is an in vivo, ex vivo or in vitro immune response.
28. The polymersome according to any one of preceding items, wherein said polymersome is capable of enhancing the frequency of effector CD4(+) T cells and CD8(+) T cells, preferably said enhancing is an in vivo, ex vivo or in vitro enhancing.
29. The polymersome according to any one of preceding items, wherein said polymersome is capable of releasing said antigen inside an endosome, preferably said endosome is a late-endosome, further preferably said releasing is an in vivo, ex vivo or in vitro releasing.
30. The polymersome according to any one of the preceding items, wherein the antigen is a self-antigen or a non-self-antigen or a neo-antigen.
31. The polymersome according to any one of the preceding items, wherein said antigen is selected from the group consisting of:
   i) a polypeptide which is at least 80% or more (e.g., at least 85%, at least 90%, at least 95%, at least 96%, at least 97%, at least 98%, at least 99% or 100%) identical to a viral polypeptide sequence; preferably said viral polypeptide sequence is Influenza hemagglutinin or Swine Influenza hemagglutinin, further preferably said viral polypeptide sequence is selected from the group consisting of: SEQ ID NO: 2, SEQ ID NO: 3, SEQ ID NO: 4 and SEQ ID NO: 5;
   ii) a polypeptide which is at least 80% or more (e.g., at least 85%, at least 90%, at least 95%, at least 96%, at least 97%, at least 98%, at least 99% or 100%) identical to a bacterial polypeptide sequence;
   iii) a polypeptide which is at least 80% or more (e.g., at least 85%, at least 90%, at least 95%, at least 96%, at least 97%, at least 98%, at least 99% or 100%) identical to a mammalian or avian polypeptide sequence, preferably said mammalian or avian polypeptide sequence is Ovalbumin (OVA), further preferably said avian polypeptide sequence has SEQ ID NO: 1.
32. The polymersome according to item 31, wherein said mammalian polypeptide sequence is selected from the group consisting of: human, rodent, rabbit and horse polypeptide sequence.
33. The polymersome according to any one of preceding items, wherein said polymersome is selected from a group consisting of: cationic, anionic and nonionic polymersome.
34. The polymersome according to any one of preceding items, wherein the polymersome has a circumferential membrane (formed by) of an amphiphilic polymer or formed by a mixture of two or more amphiphilic polymers.
35. The polymersome according to item 34, wherein the amphiphilic polymer is essentially non-immunogenic or essentially non-antigenic.
36. The polymersome according to item 34 or 35, wherein the amphiphilic polymer is neither an immunostimulant nor adjuvant.
37. The polymersome according to any of items 34 to 36, wherein the amphiphilic polymer comprises a diblock or a triblock (A-B-A or A-B-C) copolymer.
38. The polymersome according to any one of items 34 to 37, wherein the amphiphilic polymer comprises a copolymer poly(N-vinylpyrrolidone)-b-PLA.
39. The polymersome according to any one of items 34 to 38, wherein the amphiphilic polymer comprises at least one monomer unit of a carboxylic acid, an amide, an amine, an alkylene, a dialkylsiloxane, an ether or an alkylene sulphide.
40. The polymersome according to any one of items 34 to 39, wherein the amphiphilic polymer is a polyether block selected from the group consisting of an oligo(oxyethylene) block, a poly(oxyethylene) block, an oligo(oxypropylene) block, a poly(oxypropylene) block, an oligo(oxybutylene) block, a poly(oxybutylene) block, copolymer poly(2-methyl-2-oxazoline)-block-poly(dimethylsiloxane)-block-poly(2-methyl-2-oxazoline, methacrylate-terminated ABA block copolymer poly(2-methyl-2-oxazoline)-block-poly(dimethylsiloxane) block-poly(2-methyl-2-oxazoline) (MA-ABA) and mixtures thereof.
41. The polymersome according to any one of items 34 to 40, wherein the amphiphilic polymer is a poly(butadiene)-poly(ethylene oxide) (PB-PEO) diblock copolymer.
42. The polymersome according to item 41, wherein said PB-PEO diblock copolymer comprises 5-50 blocks PB and 5-50 blocks PEO.
43. The polymersome according to any one of items 34 to 42, wherein the amphiphilic polymer is a poly(lactide)-poly(ethylene oxide)/1-palmitoyl-2-oleoyl-sn-glycero-3-phospho-L-serine (PLA-PEO/POPC) copolymer, preferably said PLA-PEO/POPC has a ratio of 75 to 25 (e.g., 75/25) of PLA-PEO to POPC (e.g., PLA-PEO/POPC).
44. The polymersome according to any one of items 34 to 43, wherein said amphiphilic polymer is a poly(caprolactone)-poly(ethylene oxide)/1-palmitoyl-2-oleoyl-sn-glycero-3-phospho-L-serine (PCL-PEO/POPC) copolymer, preferably said PCL-PEO/POPC has a ratio of 75 to 25 (e.g., 75/25) of PCL-PEO to POPC (e.g., PCL-PEO/POPC).
45. The polymersome according to any one of items 34 to 44, wherein said amphiphilic polymer is polybutadiene-polyethylene oxide (BD).
46. The polymersome according to any one of items 34 to 45, wherein said polymersome comprises diblock copolymer PBD₂₁-PEO₁₄ (BD21), PBD₃₇-PEO₁₄ (BD21) and/or the triblock copolymer PMOXA₁₂-PDMS₅₅-PMOXA₁₂.
47. The polymersome according to any one of the items 1 to 46, wherein: i) said antigen is soluble or solubilized; and/or ii) the polymersome further comprises an encapsulated antigen.
48. The polymersome of any one of the items 34 to 47, wherein the two or more amphiphilic polymers have different block lengths.
49. The polymersome of item 48, wherein the polymersome comprises two different is polybutadiene-polyethylene oxide (BD) polymers, for example, a BD21 and BD37 or the polymersome, or comprises a mixture of PS-PEG block copolymer with other block PS-PIAT or PS-PEG of different block lengths.
50. The polymersome of item 48 or 49, wherein the antigen is conjugated to the amphiphilic polymer with the longer block lengths.
51. A method for producing of polymersome capable of eliciting an immune response, comprising: conjugating an antigen selected from the group consisting of:
   a) a polypeptide;
   b) a carbohydrate;
   c) a polynucleotide;
   d) a combination of (a) and/or (b) and/or (c);

to the exterior surface of the polymersome via a covalent bond.
52. A polymersome having an antigen conjugated to the exterior surface produced by the method of item 51.
53. A composition comprising a polymersome according to any one of preceding items.
54. The composition according to item 53, wherein the composition is a pharmaceutical or diagnostic composition.
55. The composition according to item 53 to 54, wherein said composition is an immunogenic, antigenic or immunotherapeutic composition.
56. The composition according to any one of items 53 to 55, further comprising one or more immunostimulants and/or one or more adjuvants.
57. The composition according to any one of items 53 to 56, wherein the composition is a vaccine.
58. The composition according to any one of items 53 to 57, formulated for intradermal, intraperitoneal, intramuscular, subcutaneous, intravenous injection, or non-invasive administration to a mucosal surface.
59. A population of isolated antigen presenting cells or a hybridoma cell exposed to the polymersome according to any of clams 1 to 52 or a composition according to any one of items 53 to 58.
60. The population of antigen presenting cells according to item 59, wherein the antigen presenting cells comprise dendritic cells.
61. The population of antigen presenting cells according to item 59 or 60, wherein the antigen presenting cells comprise macrophages.
62. The population of antigen presenting cells according to any one of items 59 to 61, wherein the antigen presenting cells comprise B-cells.
63. A vaccine comprising the polymersome of any of items 1 to 52 or the composition of items 53 to 56, and further comprising a pharmaceutically accepted excipient or carrier.
64. A kit comprising the polymersome of any of items 1 to 52 or the composition of items 53 to 58.
65. A method of eliciting an immune response in a subject comprising administering to a subject a therapeutically effective amount of said polymersome, composition, antigen presenting cells, hybridoma or vaccine as defined in any of the items 1 to 64.
66. The method of item 65, wherein the subject is a mammal or a non-mammalian animal.
67. The method of item 66, wherein the non-mammalian animal is a bird or a fish.
68. The method of item 65, wherein the mammalian animal is selected from the group consisting of a human, a rodent (e.g. a mouse or a rat), a rabbit, a pig, a cow, a sheep, a horse, a dog, and a cat.
69. The method of item 67, wherein the bird is selected from the group consisting of a chicken, a duck, a goose, and a turkey.
70. The method of any of items 65 to 69, wherein administering comprises an administration route selected from the group consisting of oral, intradermal, intraperitoneal, intramuscular, subcutaneous, intravenous injection, and non-invasive administration to a mucosal surface.
71. The method of any one of items 65 to 70, wherein the immune response is a broad immune response.
72. The method of eliciting an immune response of any one of items 65 to 71, wherein the immune response comprises a CD4(+) T cell-mediated immune response.
73. A method of treating or preventing a disease in a subject comprising administering to the subject a therapeutically effective amount of the polymersome, composition, antigen presenting cells, hybridoma or vaccine as defined in any one of the preceding items 1 to 64.
74. The method of item 73, wherein the disease is selected from the group consisting of an infectious disease, a cancer, and an autoimmune disease
75. The method of item 74, wherein the infectious disease is a viral or bacterial infectious disease.
76. A method for immunizing a non-human animal, said method comprising administering to the non-human animal the polymersome, composition, antigen presenting cells hybridoma or vaccine as defined in any one of the preceding items 1 to 64.
77. The method of item 76, wherein administering comprises an administration route selected from the group consisting of oral, intradermal, intraperitoneal, intramuscular, subcutaneous, intravenous injection, and non-invasive administration to a mucosal surface.
78. A method of preparing an antibody, comprising:
   i) immunizing a non-human mammal with the polymersome, composition, antigen presenting cells, hybridoma or vaccine as defined in any one of preceding items 1 to 64;
   ii) isolating an antibody obtained in step (i).
79. The method of item 78, wherein the antibody is a monoclonal antibody (mAb).
80. The polymersome, composition, antigen presenting cells, hybridoma or vaccine as defined in any one of preceding items 1 to 64, for use as a medicament.
81. The polymersome, composition, antigen presenting cells, hybridoma or vaccine as defined in any one of preceding items 1 to 64 for use in one or more of the following methods:
   i) in a method of antibody discovery and/or screening and/or preparation;
   ii) in a method of vaccine discovery and/or screening and/or preparation;
   iii) in a method of production or preparation of an immunogenic or immunostimulant composition;
   iv) in a method of targeted delivery of a protein and/or peptide;
   v) in a method of stimulating an immune response to an antigen, preferably said antigen is an antigen according to any one of preceding items;
   vi) in a method of delivering a peptide and/or protein to an antigen-presenting cells (APCs) according to any one of preceding items;
   vii) in a method of triggering an immune response comprising CD4(+) T cell-mediated immune response;
   viii) in a method for treatment, amelioration, prophylaxis or diagnostics of an infectious disease, preferably said infectious disease is a viral or bacterial infectious disease; further preferably said viral infectious disease is selected from a group consisting of: influenza infection, respiratory syncytial virus infection, herpes virus infection.
   ix) in a method for treatment, amelioration, prophylaxis or diagnostics of a cancer or an autoimmune disease;
   x) in a method for sensitizing cancer cells to chemotherapy;
   xi) in a method for induction of apoptosis in cancer cells;
   xii) in a method for stimulating an immune response in a subject;
   xiii) in a method for immunizing a non-human animal;
   xiv) in a method for preparation of hybridoma;
   xv) in a method according to any one of preceding items;
   xvi) in a method according to any one of preceding i)-xv), wherein said method is in vivo and/or ex vivo and/or in vitro method;
   xvii) in a method according to any one of preceding i)-xvi), wherein said antigen is heterologous to the environment in which said antigen is used.
82. Use of the polymersome, composition, antigen presenting cells, hybridoma or vaccine as defined in any one of preceding items 1 to 64 for one or more of the following:
   i) for antibody discovery and/or screening and/or preparation;
   ii) for vaccine discovery and/or screening and/or preparation;
   iii) for production or preparation of an immunogenic or immunostimulant composition;
   iv) for targeted delivery of proteins and/or peptides, preferably said targeted delivery is a targeted delivery of antigenic proteins and/or peptides; further preferably said targeted delivery is carried out in a subject;
   v) for stimulating an immune response to an antigen, preferably for use in stimulating an immune response to an antigen in a subject;
   vi) for delivering a peptide or protein to an antigen-presenting cell (APC); preferably said peptide or protein is an antigen, further preferably said peptide or protein is immunogenic or immunotherapeutic;
   vii) for triggering an immune response comprising CD4(+) T cell-mediated immune response;
   viii) in a method for treatment, amelioration, prophylaxis or diagnostics of an infectious disease, preferably said infectious disease is a viral or bacterial infectious disease; further preferably said viral infectious disease is selected from a group consisting of: influenza infection, respiratory syncytial virus infection; herpes virus infection;
   ix) for treatment, amelioration, prophylaxis or diagnostics of a cancer or an autoimmune disease;
   x) for sensitizing cancer cells to chemotherapy;
   xi) for induction of apoptosis in cancer cells;
   xii) for stimulating an immune response in a subject;
   xiii) for immunizing a non-human animal;
   xiv) for preparation of hybridoma;
   xv) in a method according to any one of preceding items;
   xvi) for use according to any one of preceding i)-xv), wherein said use is in vivo and/or ex vivo and/or in vitro use;
   xvii) for use according to any one of preceding i)-xvi), wherein said antigen is heterologous to the environment in which said antigen is used.
83. The use of the polymersome according to any one of preceding items 1 to 52, wherein said polymersome having a diameter of about 100 nm or more and comprising a conjugated or attached antigen, wherein said conjugated or attached antigen is selected from the group consisting of:
   i) a polypeptide;
   ii) a carbohydrate;
   iii) a polynucleotide, or
   iv) a combination of i) and/or ii) and/or iii)

for eliciting an immune response.
84. The use of item 83, wherein the diameter of the polymersome is in the range of from about 100 nm to 1 μm, or from about 140 nm to about 1 μm, or from about 140 nm to about 750 nm, or from about 140 nm to about 500 nm, or from about 140 nm to about 250 nm, from about 140 nm to about 240 nm, from about 150 nm to about 235 nm, from about 170 nm to about 230 nm, or from about 220 nm to about 180 nm, or from about 190 nm to about 210 nm.
85. The use of a collection of polymersomes as defined in any one of item 1 to 52, having a mean diameter of about 100 nm or more, 110 nm or more, 120 nm or more, 130 nm or more, 140 nm or more, the polymersomes of the collection comprising a conjugated or attached antigen, wherein said conjugated or attached antigen is selected from the group consisting of:
   i) a polypeptide;
   ii) a carbohydrate;
   iii) a polynucleotide, or
   iv) a combination of i) and/or ii) and/or iii)

for eliciting an immune response.
86. The use of item 83, wherein the diameter of the polymersome is in the range of from about 100 nm to 1 μm, or from about 140 nm to about 1 μm, or from about 140 nm to about 750 nm, or from about 140 nm to about 500 nm, or from about 140 nm to about 250 nm, from about 140 nm to about 240 nm, from about 150 nm to about 235 nm, from about 170 nm to about 230 nm, or from about 220 nm to about 180 nm, or from about 190 nm to about 210 nm.
87. The use according to any one of items 83 to 86, wherein the polymersome is selected from a group consisting of: cationic, anionic and nonionic polymersome.
88. The use according to any one of items 83 to 87, wherein the polymersome has a circumferential membrane (formed by) of an amphiphilic polymer.
89. The use of a polymersome according to item 88, wherein the amphiphilic polymer is essentially non-immunogenic or essentially non-antigenic.
90. The use of a polymersome according to item 87 or 88, wherein the amphiphilic polymer is neither an immunostimulant nor adjuvant.
91. The use of a polymersome according to any of items 88 to 90, wherein the amphiphilic polymer comprises a diblock or a triblock (A-B-A or A-B-C) copolymer.
92. The use of a polymersome according to any one of items 88 to 91, wherein the amphiphilic polymer comprises a copolymer poly(N-vinylpyrrolidone)-b-PLA.
93. The use of a polymersome according to any one of items 88 to 92, wherein the amphiphilic polymer comprises at least one monomer unit of a carboxylic acid, an amide, an amine, an alkylene, a dialkylsiloxane, an ether or an alkylene sulphide.
94. The use of a polymersome according to any one of items 88 to 93, wherein the amphiphilic polymer is a polyether block selected from the group consisting of an oligo(oxyethylene) block, a poly(oxyethylene) block, an oligo(oxypropylene) block, a poly(oxypropylene) block, an oligo(oxybutylene) block and a poly(oxybutylene) block.
95. The use of a polymersome according to any one of items 88 to 94, wherein the amphiphilic polymer is a poly(butadiene)-poly(ethylene oxide) (PB-PEO) diblock copolymer.
96. The use of a polymersome according to item 95, wherein said PB-PEO diblock copolymer comprises 5-50 blocks PB and 5-50 blocks PEO.
97. The use of a polymersome according to any one of items 88 to 96, wherein the amphiphilic polymer is a poly(lactide)-poly(ethylene oxide)/1-palmitoyl-2-oleoyl-sn-glycero-3-phospho-L-serine (PLA-PEO/POPC) copolymer, preferably said PLA-PEO/POPC has a ratio of 75 to 25 (e.g., 75/25) of PLA-PEO to POPC (e.g., PLA-PEO/POPC).
98. The use of a polymersome according to any one of items 88 to 97, wherein said amphiphilic polymer is a poly(caprolactone)-poly(ethylene oxide)/1-palmitoyl-2-oleoyl-sn-glycero-3-phospho-L-serine (PCL-PEO/POPC) copolymer, preferably said PCL-PEO/POPC has a ratio of 75 to 25 (e.g., 75/25) of PCL-PEO to POPC (e.g., PCL-PEO/POPC).
99. The use of a polymersome according to any one of items 88 to 98, wherein said amphiphilic polymer is polybutadiene-polyethylene oxide (BD).
100. The use of polymersome according to any one of items 88 to 99, wherein said polymersome comprises diblock copolymer PBD₂₁-PEO₁₄ (BD21) and/or the triblock copolymer PMOXA₁₂-PDMS₅₅-PMOXA₁₂.
101. The polymersome according to any one of the preceding items 1 to 52, wherein the polymersome further comprises an encapsulated antigen.

### EXAMPLES OF THE INVENTION

In order that the invention may be readily understood and put into practical effect, some aspects of the invention are described by way of the following non-limiting examples.

### Materials and Methods

Conjugation of BD21 Vesicles to Ovalbumin (OVA, SEQ ID NO: 1):

BD₂₁+5% DSPE-PEG(3000)-Maleimide Vesicles Formation:

100 μL of BD₂₁ (100 mg/mL) in CHCl₃ was transferred to 25 mL of single-neck RBF (round bottom flask) to which was added 80.89 μL of DSPE-PEG-Maleimide (10 mg/mL in CHCl₃). The solvent was slowly evaporated under reduced pressure at 35° C. to get wide-spread thin-film and was dried in desiccator under vacuum for 6 hours. 1 mL of NaHCO₃ buffer (10 mM, 0.9% NaCl, pH 6.5) was added to the thin-film for rehydration and stirred at 25° C. for 16-20 hours to form milky homogeneous solution. After rehydration for 16-20 hours, the solution was extruded with 200 nm Whatman membrane at 25° C. for 21 times. The solution was transferred to dialysis bag (MWCO (weight cut-off): 300 KD) and dialyzed in NaHCO₃ buffer (10 mM, 0.9% NaCl, pH 6.5) (2×500 mL and 1×1 L; first two dialysis were done for 3 hours each and the last one for 16 hours). Vesicle size and mono-dispersity was characterized by dynamic light scattering Instrument (Malvern, United Kingdom) (100× dilution with 1× PBS).

Conjugation of BD₂₁+DSPE-PEG(3000)-maleimide (5%) to OVA:

OVA (0.5 mg) was dissolved in 200 μL of NaHCO₃ buffer (10 mM, 0.9% NaCl, pH 6.5) to which was added 2.5 mg of TCEP-HCl (dissolved in 100 μL of same NaHCO₃ buffer) and incubated for 20 minutes. pH of the reaction was adjusted from ˜2.0 to 6-7 using 1N NaOH solution (˜10 μL). 350 μL of polymersomes (10 mg/mL of BD/DSPE-PEG(3000)-Maleimide 5% in 10 Mm NaHCO₃, 0.9% NaCl buffer, pH 7.0) was then added to the protein mix and pH of the reaction was adjusted again to pH 7.0 (if pH of reaction was not 7). Reaction was incubated at 24° C. for 3 hours away from light. The reaction solution (˜660 μL) was transferred to dialysis bag (MWCO: 1000 KD) and dialyzed in NaHCO₃ buffer (10 mM, 0.9% NaCl, pH 7.0) (3×1 L; first two dialysis were done for 3 hours each and the last one for 16 hours). 100 μL of dialyzed solution was purified through SEC chromatography and collected in 96-well plate. The corresponding ACM peak fractions were combined and lyophilized for quantification by SDS-PAGE.

For comparison, OVA was also encapsulated in BD₂₁ alone. For this a film was produced as above using 100 μl of a 100 mg/ml BD₂₁ stock dissolved in CHCl₃. Rehydration was then performed by adding 1 mL solution of 0.5 mg/ml solubilized OVA protein in 1× PBS buffer. The mixture was stirred at 600 rpm, 4° C. for at least 18 hours to allow the formation of polymer vesicles, extruded and dialyzed as above.

Conjugation of BD₂₁ Vesicles to Hemagglutinin (HA, SEQ ID NO: 4):

Preparation of BD₂₁-CHO from BD₂₁:

To a stirred solution of BD₂₁ (100 mg) in single-neck RBF was dissolved in anhydrous CH₂Cl₂ (6 mL) and was added Dess-Martin periodinane (10 mg, 0.4 equiv) at 0° C. in one-portion. Reaction was stirred at 25° C. for 4 hours. Then 1:1 mixture of saturated NaHCO₃ and Na₂S₂O₃ (20 mL) was added and stirred at the same temperature for 2 hours. Organic layers was separated, and the aqueous layers was extracted with CH₂Cl₂ (20 mL) and separated the organic layer. The combined organic layers were washed with 1:1 mixture of sat. NaHCO₃ and Na₂S₂O₃ (20 mL), brine (20 mL), dried over anhydrous Na₂SO₄ and evaporated under reduced pressure to get colourless viscous oil (100 mg, quantitative). Modification yield was estimated to be around 30% by NMR.

Conjugation of BD-CHO to HA:

10 mg of modified BD₂₁-CHO (colourless viscous oil) was dissolved in 0.5 mL of CHCl₃ in 25 mL of single-neck RBF and slowly evaporated the solvent under reduced pressure using Rotavap at 35° C. for 10 minutes to get wide spread thin-film. The film was dried under vacuum in desiccator for 6 hours. The film was rehydrated in 400 μl of borate buffer (borate 10 mM, 150 mM NaCl, pH 7.5) for 30 minutes before adding 0.5 mg of HA (150 μl of HA was prepared by pre-equilibrating it in borate buffer by dialysis). Reaction was stirred at 25° C. for 16 hours. 20 μL of NaCNBH₄ was then added to the solution (preparation: 126 mg of NaCNBH₄ was dissolved in 1 mL of Millipore water and degassed the excess H₂ gas by stirring the solution at 25° C. for 30 minutes) and kept on stirring at 25° C. for another 8-16 hours. The conjugated polymersomes were extruded by using 200 nm Whatman membrane at 25° C. for 21 times. The reaction solution was transferred to dialysis bag (MWCO: 1000 KD) and dialysed in PBS buffer (1×, pH 7.4) (3×1 L; first two dialysis were done for 3 hours each and the last one for 16 hours). After dialysis, 400 μL of dialysed solution was purified through SEC chromatography (Size-exclusion chromatography) and collected in a 96-well plate. The presence of coupled HA was detected using both Western Blot and ELISA assays (Enzyme-linked Immunosorbent Assay). Vesicle size and mono-dispersity was characterized by dynamic light scattering (100× dilution with 1× PBS).

For comparison, HA was also encapsulated in BD₂₁ alone. For this a film was produced as above using 100 μl of a 100 mg/ml BD₂₁ stock dissolved in CHCl₃. Rehydration was then performed by adding 1 mL solution containing 20 μg of HA protein in 1× PBS buffer. The mixture was stirred at 600 rpm, 4° C. for at least 18 hours to allow the formation of polymer vesicles, extruded and dialyzed as above.

Quantification of Coupled HA and OVA:

To detect the presence of coupled proteins several techniques were used. 100 to 300 μl of dialyzed sample was loaded onto a Size Exclusion Chromatography (SEC, Akta) using a Sephacryl column. SEC fractions corresponding to the peak of ACM vesicles were pooled or used as is to either be analysed by SDS-PAGE or/and ELISA. For SDS-PAGE, 20-40 μl of each fraction was mixed DMSO (20% v/v) and vortexed thoroughly before adding loading buffer. Different amounts of free BSA (Bovine serum albumin), HA or OVA was added for quantification. After migration, the gel was either stained by sliver staining (OVA) or used for a membrane transfer and immunoblotting with rabbit polyclonal antibody (HA). To further ensure that HA was coupled to the polymer, 25 ul of all SEC fractions was coated into a Maxisorp 384-well plate overnight at 4° C. After blocking with 3% BSA, rabbit polyclonal anti-HA antibody was used as primary antibody followed by HRP (horseradish peroxidase) coupled anti-rabbit as secondary. TMB substrate was added and reaction was stopped using 1M HCl. Optical densities were quantified at 450 nm.

Mouse Immunizations and Titer Determination (mAb):

C57bl/6 mice were immunized with different OVA formulations: PBS (negative control), free OVA with or without Sigma Adjuvant System (SAS), OVA encapsulated ACMs or OVA conjugated ACMs. Balb/c mice were immunized with different HA formulations: PBS (negative control), free HA, HA encapsulated ACMs or HA conjugated ACMs. Both trials were performed by doing a prime and a boost 21 days later. All immunizations were performed with a same final amount of antigen within each trial: 5-10 μg OVA/injection/mouse or 100-200 ng HA/injection/mouse. Final bleeds were collected 42 days after prime. ELISA was then performed to assess titers: OVA or HA were coated onto MaxiSorp plates (1 μg/ml in carbonate buffer) overnight. Plates were blocked using 3% BSA in PBS for 1 h at RT. All sera were diluted at 1:100 and incubated on plates for 1 h at RT. After 3 washes with PBS+0.05% Tween 20, secondary antibody anti-mouse IgG HRP coupled was incubated at 1:10,000 dilution for 1 h, RT (room temperature). After 3 washes with PBS/Tween 20 buffer, TMB substrate was added and reaction was stopped using 1M HCl. Optical densities were quantified at 450 nm.

### Results

### Example 1

### ACM Polymersomes Coupling to OVA

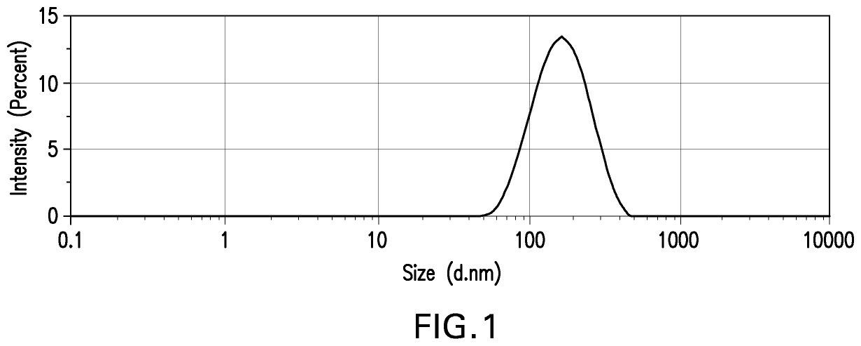

Polymersomes (also called ACMs (artificial cell membranes) prepared with 5% DSPE-PEG(3000)-Maleimide were used to couple OVA through available cysteines. At least one cysteine has been shown to be accessible to solvent (Tatsumi et al., 1997). Coupling conditions were achieved in pH-controlled environment. FIG. 1 shows the Dynamic Light Scattering (DLS) profile from OVA coupled polymersomes which is matching standard features of these exemplary polymersomes of the invention (average (mean) size of the population/collection of polymersomes: 152 nm; pdi: 0.229).

After extensive dialysis, 100 μl of sample was separated using SEC (FIG. 2A) and 48 fractions of around 180 μl were collected. Pooled fractions corresponding to the peak were lyophilized and resuspended into 500 μl. 20 ul was loaded onto an SDS-PAGE together with some BSA standards (FIG. 2B). A band at the size corresponding to OVA protein was detected suggesting that OVA was successfully coupled to ACMs vesicles. The amount of coupled OVA was estimated to be around 20 μg/ml. Notably, the BD₂₁ coupling to OVA protein did not modify its migration properties as seen for HA (see below). This is probably due to the fact that OVA is likely to be modified only at one cysteine residue per OVA protein, all other five cysteines being either buried or engaged in a disulfide bound.

### Example 2

### BD₂₁-CHO Polymersomes Coupling to HA

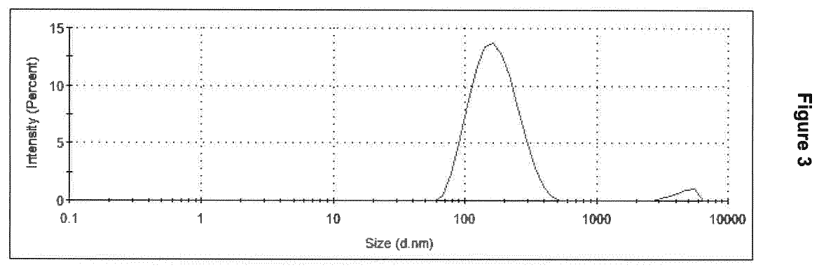

BD₂₁ polymer was modified as described in the methods and the aldehyde modification percentage was estimated to be around 30-40% by NMR. The aldehyde moiety added to the BD₂₁ will react with the primary amines of HA's lysine and arginine residues. After overnight coupling followed by extensive dialysis, the resulting vesicles were characterized. DLS showed a slightly smaller size (average size: 104 nm) and acceptable pdi (pdi: 0.191) (FIG. 3).

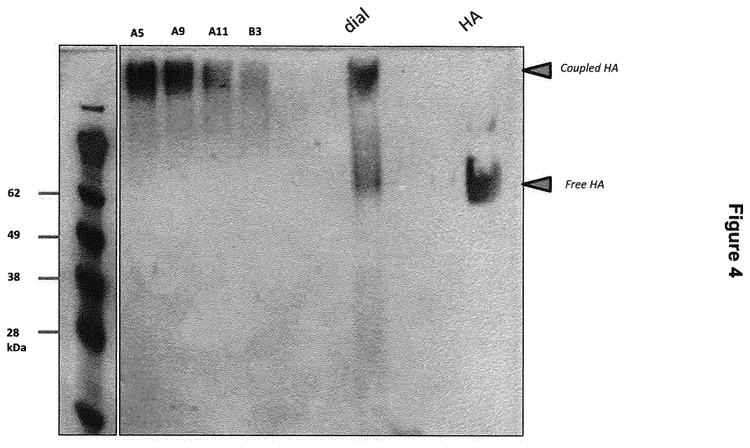

400 μl of the final product were separated by SEC as above (see FIG. 4, light gray trace). Fractions corresponding to the peak were loaded individually onto an SDS-PAGE followed by membrane transfer for immunoblotting. A band with a high molecular weight was detected and seemed to decrease in later fractions outside the peak suggesting that this band corresponds to the conjugated HA. The observed high molecular weight could be due to the numerous polymer molecules coupled to the HA increasing its molecular weight. In addition, covalently bound polymer could partially compete with the binding of SDS of the loading buffer decreasing the final charges state compared to free HA. With a lower negative charge, one would expect conjugated HA protein to migrate less which would result in an apparent higher molecular weight. The dialyzed sample (non-separated on SEC) shows residual free HA probably coming from aggregated HA that could not be dialyzed. Concentration of conjugated HA was determined to around 1 μg/ml.

To ascertain that HA proteins are accessible at the surface of particles, ELISA was conducted on all collected fractions coated overnight on a Maxisorp plate able to trap BD₂₁ vesicles. HA protein was clearly detected and when the ELISA profile was superimposed on the SEC, both profiles nicely correlated (FIG. 4, black trace) confirming that HA was coupled to BD₂₁ and was accessible to an antibody detection.

### Example 3

### Immunizations and Sera Tittering

C57bl/6 mice were immunized with the following formulations: a negative control (PBS), free OVA with or without Sigma Adjuvant System (SAS), BD₂₁ encapsulated OVA and BD₂₁ conjugated OVA. All immunizations had a same amount of 4 μg of OVA per injection and per mouse. 21 days after the boost, sera were collected for tittering by ELISA. Free OVA with or without adjuvant was not able to elicit an IgG response. Interestingly, at similar dose conjugated OVA was able to trigger a lot stronger titer response than encapsulated OVA.

Balb/c mice were immunized with the following formulations: a negative control (PBS), free HA, BD₂₁ encapsulated HA and BD₂₁ conjugated HA. Since some residual free HA was observable in the HA conjugated polymersome sample even after extensive dialysis, pooled fractions of SEC were used for immunizations. All immunizations had a same amount of 100-200 ng of HA per injection and per mouse. Free HA was not able to elicit an IgG response which was expected given the low amount of HA injected. Conjugated HA was able to trigger only a slightly higher response than encapsulated HA in this case.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A polymersome capable of eliciting an immune response, comprising:
i) an antigen selected from the group consisting of:
a) a polypeptide;
b) a carbohydrate;
c) a polynucleotide;
d) a combination of (a) and/or (b) and/or (c);
wherein the antigen is conjugated to the exterior surface of the polymersome via a covalent bond.

2. The polymersome of claim 1, wherein the covalent bond comprises: i) an amide moiety; and/or ii) a secondary amine moiety; and/or iii) a 1,2,3-triazole moiety, preferably said 1,2,3-triazole moiety is a 1,4-disubstituted[1,2,3]triazole moiety or a 1,5-disubstituted[1,2,3]triazole moiety; and/or iv) pyrazoline moiety, and/or vi) ester moiety; and/or vii) carbamate and or carbonate moiety.

3. The polymersome of claim 2, wherein the covalent bond that conjugates the antigen to the exterior surface of the polymersome is formed by reacting a reactive group present on the exterior surface of the polymersome with a reactive group of the antigen.

4. The polymersome of claim 3, wherein the covalent bond is selected from the group consisting of: i) a carboxamide bond; ii) a 1,4-disubstituted[1,2,3]triazole or 1,5-disubstituted[1,2,3]triazole bond; iii) a substituted pyrazoline bond.

5. The polymersome of claim 4, wherein: i) the reactive group present on the exterior surface of the polymersome is an aldehyde group and the reactive group of the antigen is an amine group, thereby forming the carboxamide group; or ii) the reactive group present on the exterior surface of the polymersome is an alkyne group and the reactive group of the antigen is an azide group, thereby forming the 1,2,3-triazole group, preferably via copper- or ruthenium catalyzed azide-alkyne cycloaddition, further preferably said 1,2,3-triazole is 1,4-disubstituted or 1,5-disubstituted; or iii) the reactive group present on the exterior surface of the polymersome is a methacrylate- and/or hydroxyl group and the reactive group of the antigen is a tetrazole group, thereby forming the pyrazoline group, preferably said forming of the pyrazoline group comprises a nitrile imine intermediate.

6. The polymersome of claim 5, wherein the carboxamide bond has further been reacted with a reducing agent to form a secondary amine.

7. The polymersome of any of the foregoing claims, wherein the covalent bond is formed via a linker moiety.

8. The polymersome of claim 7, wherein the linker moiety L is a peptidic linker or a straight or branched hydrocarbon-based linker.

9. The adapter molecule of claim 7 or 8, wherein the linker moiety comprises 1 to about 550 main chain atoms, 1 to about 500 main chain atoms, 1 to about 450 main chain atoms, 1 to about 350 main chain atoms, 1 to about 300 main chain atoms, 1 to about 250 main chain atoms, 1 to about 200 main chain atoms, 1 to about 150 main chain atoms, 1 to about 100 main chain atoms, 1 to about 50 main chain atoms, 1 to about 30 main chain atoms, 1 to about 20 main chain atoms, 1 to about 15 main chain atoms, or 1 to about 12 main chain atoms, or 1 to about 10 main chain atoms, wherein the main chain atoms are carbon atoms that are optionally replaced by one or more heteroatoms selected from the group consisting of N, O, P and S.

10. The polymersome of any of claims 7 to 9, wherein the linker moiety comprise a membrane anchoring domain which integrates the linker moiety into the membrane of the polymersome.

11. The polymersome of claim 10, wherein the membrane anchoring domain comprises a lipid.

12. The polymersome of claim 11, wherein the lipid is a phospholipid ora glycolipid.

13. The polymersome of claim 12 wherein the glycolipid comprises glycophosphatidylinositol (GPI).

14. The polymersome of claim 12, wherein the phospholipid is a phosphosphingolipid or a glycerophospholipid.

15. The polymersome of claim 12, wherein the phosphosphingolipid comprises distearoylphosphatidylethanolamine [DSPE] conjugate to polyethylene glycol (PEG) (DSPE-PEG) or a cholesterol based conjugate.

16. The polymersome of claim 15, wherein the DSPE-PEG comprises from 2 to about 500 ethylene oxide units.

17. The polymersome of any of the foregoing claims, wherein the linker is non-hydrolysable and/or non-oxidizable under physiological conditions.

18. The polymersome according to any one of preceding claims, wherein the physiological conditions are characterized by: a temperature in the range of about 20-40° C., atmospheric pressure of 1 and pH in the range of about 6-8.

19. The polymersome according to any one of preceding claims, wherein the polymersome has a vesicular morphology.

20. The polymersome according to any one of preceding claims, wherein the elicited immune response comprises a humoral immune response and/or a cellular immune response.

21. The polymersome according to any one of preceding claims, wherein the polymersome is an oxidation-stable polymersome.

22. The polymersome according to any one of preceding claims, wherein the polymersome has a diameter greater than 70 nm, preferably said diameter ranging from about 100 nm to about 1 μm, or from about 100 nm to about 750 nm, or from about 100 nm to about 500 nm, or from about 125 nm to about 250 nm, from about 140 nm to about 240 nm, from about 150 nm to about 235 nm, from about 170 nm to about 230 nm, or from about 220 nm to about 180 nm, or from about 190 nm to about 210 nm.

23. The polymersome according to any one of preceding claims, wherein the polymersome is oxidation-stable in the presence of serum components, preferably said oxidation-stability is an in vivo, ex vivo or in vitro oxidation-stability.

24. The polymersome according to any one of preceding claims, wherein the polymersome is stable inside an endosome, preferably said stability is an in vivo, ex vivo or in vitro stability.

25. The polymersome according to any one of preceding claims, wherein the polymersome has an improved oxidation stability compared to corresponding oxidation stability of a liposome or nanoparticle, preferably said improved stability is an in vivo, ex vivo or in vitro improved stability.

26. The polymersome according to any one of preceding claims, wherein the polymersome is capable of releasing said antigen in an oxidation-independent manner and triggering a humoral immune response, wherein said releasing is an in vivo, ex vivo or in vitro releasing.

27. The polymersome according to any one of preceding claims, wherein said humoral immune response comprises production of specific antibodies, further preferably said immune response is an in vivo, ex vivo or in vitro immune response.

28. The polymersome according to any one of preceding claims, wherein said polymersome is capable of enhancing the frequency of effector CD4(+) T cells and/or CD8(+)T cells, preferably said enhancing is an in vivo, ex vivo or in vitro enhancing.

29. The polymersome according to any one of preceding claims, wherein said polymersome is capable of releasing said antigen inside an endosome, preferably said endosome is a late-endosome, further preferably said releasing is an in vivo, ex vivo or in vitro releasing.

30. The polymersome according to any one of the preceding claims, wherein the antigen is a self-antigen, including a neoantigen, or a non-self-antigen.

31. The polymersome according to any one of the preceding claims, wherein said antigen is selected from the group consisting of:
i) a polypeptide which is at least 80% or more (e.g., at least 85%, at least 90%, at least 95%, at least 96%, at least 97%, at least 98%, at least 99% or 100%) identical to a viral polypeptide sequence; preferably said viral polypeptide sequence is Influenza hemagglutinin or Swine Influenza hemagglutinin;
ii) a polypeptide which is at least 80% or more (e.g., at least 85%, at least 90%, at least 95%, at least 96%, at least 97%, at least 98%, at least 99% or 100%) identical to a bacterial polypeptide sequence;
iii) a polypeptide which is at least 80% or more (e.g., at least 85%, at least 90%, at least 95%, at least 96%, at least 97%, at least 98%, at least 99% or 100%) identical to a mammalian or avian polypeptide sequence, preferably said mammalian or avian polypeptide sequence is Ovalbumin (OVA).

32. The polymersome according to claim 31, wherein said mammalian polypeptide sequence is selected from the group consisting of: human, rodent, rabbit and horse polypeptide sequence.

33. The polymersome according to any one of preceding claims, wherein said polymersome is selected from a group consisting of: cationic, anionic and nonionic polymersome.

34. The polymersome according to any one of preceding claims, wherein the polymersome has a circumferential membrane (formed by) of an amphiphilic polymer or formed by a mixture of two or more amphiphilic polymers.

35. The polymersome according to claim 34, wherein the amphiphilic polymer is essentially non-immunogenic or essentially non-antigenic.

36. The polymersome according to claim 34 or 35, wherein the amphiphilic polymer is neither an immunostimulant nor adjuvant.

37. The polymersome according to any of claims 34 to 36, wherein the amphiphilic polymer comprises a diblock or a triblock (A-B-A or A-B-C) copolymer.

38. The polymersome according to any one of claims 34 to 37, wherein the amphiphilic polymer comprises a copolymer poly(N-vinylpyrrolidone)-b-PLA.

39. The polymersome according to any one of claims 34 to 38, wherein the amphiphilic polymer comprises at least one monomer unit of a carboxylic acid, an amide, an amine, an alkylene, a dialkylsiloxane, an ether or an alkylene sulphide.

40. The polymersome according to any one of claims 34 to 39, wherein the amphiphilic polymer is a polyether block selected from the group consisting of an oligo(oxyethylene) block, a poly(oxyethylene) block, an oligo(oxypropylene) block, a poly(oxypropylene) block, an oligo(oxybutylene) block, a poly(oxybutylene) block, copolymer poly(2-methyl-2-oxazoline)-block-poly(dimethylsiloxane)-block-poly(2-methyl-2-oxazoline, methacrylate-terminated ABA block copolymer poly(2-methyl-2-oxazoline)-block-poly(dimethylsiloxane) block-poly(2-methyl-2-oxazoline) (MA-ABA) and mixtures thereof.

41. The polymersome according to any one of claims 34 to 40, wherein the amphiphilic polymer is a poly(butadiene)-poly(ethylene oxide) (PB-PEO) diblock copolymer.

42. The polymersome according to claim 41, wherein said PB-PEO diblock copolymer comprises 5-50 blocks PB and 5-50 blocks PEO.

43. The polymersome according to any one of claims 34 to 42, wherein the amphiphilic polymer is a poly(lactide)-poly(ethylene oxide)/1-palmitoyl-2-oleoyl-sn-glycero-3-phospho-L-serine (PLA-PEO/POPC) copolymer, preferably said PLA-PEO/POPC has a ratio of 75 to 25 (e.g., 75/25) of PLA-PEO to POPC (e.g., PLA-PEO/POPC).

44. The polymersome according to any one of claims 34 to 43, wherein said amphiphilic polymer is a poly(caprolactone)-poly(ethylene oxide)/1-palmitoyl-2-oleoyl-sn-glycero-3-phospho-L-serine (PCL-PEO/POPC) copolymer, preferably said PCL-PEO/POPC has a ratio of 75 to 25 (e.g., 75/25) of PCL-PEO to POPC (e.g., PCL-PEO/POPC).

45. The polymersome according to any one of claims 34 to 44, wherein said amphiphilic polymer is polybutadiene-polyethylene oxide (BD).

46. The polymersome according to any one of claims 34 to 45, wherein said polymersome comprises diblock copolymer PBD₂₁-PEO₁₄ (BD21), PBD₃₇-PEO₁₄ (BD21) and/or the triblock copolymer PMOXA₁₂-PDMS₅₅-PMOXA₁₂.

47. The polymersome according to any one of the claims 1 to 46, wherein: i) said antigen is soluble or solubilized; and/or ii) the polymersome further comprises an encapsulated antigen.

48. The polymersome of any one of the claims 34 to 47, wherein the two or more amphiphilic polymers have different block lengths.

49. The polymersome of claim 48, wherein the polymersome comprises two different is polybutadiene-polyethylene oxide (BD) polymers, for example, a BD21 and BD37 or the polymersome, or comprises a mixture of PS-PEG block copolymer with other block PS-PIAT or PS-PEG of different block lengths.

50. The polymersome of claim 48 or 49, wherein the antigen is conjugated to the amphiphilic polymer with the longer block lengths.

51. A method for producing a polymersome capable of eliciting an immune response, comprising:
conjugating an antigen selected from the group consisting of:
a) a polypeptide;
b) a carbohydrate;
c) a polynucleotide;
d) a combination of (a) and/or (b) and/or (c);
to the exterior surface of the polymersome via a covalent bond.

52. A polymersome having an antigen conjugated to the exterior surface produced by the method of claim 51.

53. A composition comprising a polymersome according to any one of preceding claims.

54. The composition according to claim 53, wherein the composition is a pharmaceutical or diagnostic composition.

55. The composition according to claims 53 to 54, wherein said composition is an immunogenic, antigenic or immunotherapeutic composition.

56. The composition according to any one of claims 53 to 55, further comprising one or more immunostimulants and/or one or more adjuvants.

57. The composition according to any one of claims 53 to 56, wherein the composition is a vaccine.

58. The composition according to any one of claims 53 to 57, formulated for intradermal, intraperitoneal, intramuscular, subcutaneous, intravenous injection, or non-invasive administration to a mucosal surface.

59. A population of isolated antigen presenting cells or a hybridoma cell exposed to the polymersome according to any of clams 1 to 52 or a composition according to any one of claims 53 to 58.

60. The population of antigen presenting cells according to claim 59, wherein the antigen presenting cells comprise dendritic cells.

61. The population of antigen presenting cells according to claim 59 or 60, wherein the antigen presenting cells comprise macrophages.

62. The population of antigen presenting cells according to any one of claims 59 to 61, wherein the antigen presenting cells comprise B-cells.

63. A vaccine comprising the polymersome of any of claims 1 to 52 or the composition of claims 53 to 56, and further comprising a pharmaceutically accepted excipient or carrier.

64. A kit comprising the polymersome of any of claims 1 to 52 or the composition of claims 53 to 58.

65. A method of eliciting an immune response in a subject comprising administering to a subject a therapeutically effective amount of said polymersome, composition, antigen presenting cells, hybridoma or vaccine as defined in any of the claims 1 to 64.

66. The method of claim 65, wherein the subject is a mammal or a non-mammalian animal.

67. The method of claim 66, wherein the non-mammalian animal is a bird.

68. The method of claim 65, wherein the mammalian animal is selected from the group consisting of a human, a rodent, a mouse, a rat, a pig, a cow, a sheep, a horse, a dog, and a cat.

69. The method of claim 67, wherein the bird is selected from the group consisting of a chicken, a duck, a goose, and a turkey.

70. The method of any of claims 65 to 69, wherein administering comprises an administration route selected from the group consisting of oral, intradermal, intraperitoneal, intramuscular, subcutaneous, intravenous injection, and non-invasive administration to a mucosal surface.

71. The method of any one of claims 65 to 70, wherein the immune response is a broad immune response.

72. The method of eliciting an immune response of any one of claims 65 to 71, wherein the immune response comprises a CD4(+) T cell-mediated immune response.

73. A method of treating or preventing a disease in a subject comprising administering to the subject a therapeutically effective amount of the polymersome, composition, antigen presenting cells, hybridoma or vaccine as defined in any one of the preceding claims 1 to 64.

74. The method of claim 73, wherein the disease is selected from the group consisting of an infectious disease, a cancer, and an autoimmune disease

75. The method of claim 74, wherein the infectious disease is a viral or bacterial infectious disease.

76. A method for immunizing a non-human animal, said method comprising administering to the non-human animal the polymersome, composition, antigen presenting cells hybridoma or vaccine as defined in any one of the preceding claims 1 to 64.

77. The method of claim 76, wherein administering comprises an administration route selected from the group consisting of oral, intradermal, intraperitoneal, intramuscular, subcutaneous, intravenous injection, and non-invasive administration to a mucosal surface.

78. A method of preparing an antibody, comprising:
i) immunizing a non-human mammal with the polymersome, composition, antigen presenting cells, hybridoma or vaccine as defined in any one of preceding claims 1 to 64;
ii) isolating an antibody obtained in step (i).

79. The method of claim 78, wherein the antibody is a monoclonal antibody (mAb).

80. The polymersome, composition, antigen presenting cells, hybridoma or vaccine as defined in any one of preceding claims 1 to 64, for use as a medicament.

81. The polymersome, composition, antigen presenting cells, hybridoma or vaccine as defined in any one of preceding claims 1 to 64 for use in one or more of the following methods:
i) in a method of antibody discovery and/or screening and/or preparation;
ii) in a method of vaccine discovery and/or screening and/or preparation;
iii) in a method of production or preparation of an immunogenic or immunostimulant composition;
iv) in a method of targeted delivery of a protein and/or peptide;
v) in a method of stimulating an immune response to an antigen, preferably said antigen is an antigen according to any one of preceding claims;
vi) in a method of delivering a peptide and/or protein to an antigen-presenting cells (APCs) according to any one of preceding claims;
vii) in a method of triggering an immune response comprising CD4(+) T cell-mediated immune response;
viii) in a method for treatment, amelioration, prophylaxis or diagnostics of an infectious disease, preferably said infectious disease is a viral or bacterial infectious disease; further preferably said viral infectious disease is selected from a group consisting of: influenza infection, respiratory syncytial virus infection, herpes virus infection.
ix) in a method for treatment, amelioration, prophylaxis or diagnostics of a cancer or an autoimmune disease;
x) in a method for sensitizing cancer cells to chemotherapy;
xi) in a method for induction of apoptosis in cancer cells;
xii) in a method for stimulating an immune response in a subject;
xiii) in a method for immunizing a non-human animal;
xiv) in a method for preparation of hybridoma;
xv) in a method according to any one of preceding claims;
xvi) in a method according to any one of preceding i)-xv), wherein said method is in vivo and/or ex vivo and/or in vitro method;
xvii) in a method according to any one of preceding i)-xvi), wherein said antigen is heterologous to the environment in which said antigen is used.

82. Use of the polymersome, composition, antigen presenting cells, hybridoma or vaccine as defined in any one of preceding claims 1 to 64 for one or more of the following:
i) for antibody discovery and/or screening and/or preparation;
ii) for vaccine discovery and/or screening and/or preparation;
iii) for production or preparation of an immunogenic or immunostimulant composition;
iv) for targeted delivery of proteins and/or peptides, preferably said targeted delivery is a targeted delivery of antigenic proteins and/or peptides; further preferably said targeted delivery is carried out in a subject;
v) for stimulating an immune response to an antigen, preferably for use in stimulating an immune response to an antigen in a subject;
vi) for delivering a peptide or protein to an antigen-presenting cell (APC); preferably said peptide or protein is an antigen, further preferably said peptide or protein is immunogenic or immunotherapeutic;
vii) for triggering an immune response comprising CD4(+) T cell-mediated immune response;
viii) in a method for treatment, amelioration, prophylaxis or diagnostics of an infectious disease, preferably said infectious disease is a viral or bacterial infectious disease; further preferably said viral infectious disease is selected from a group consisting of: influenza infection, respiratory syncytial virus infection; herpes virus infection;
ix) for treatment, amelioration, prophylaxis or diagnostics of a cancer or an autoimmune disease;
x) for sensitizing cancer cells to chemotherapy;
xi) for induction of apoptosis in cancer cells;
xii) for stimulating an immune response in a subject;
xiii) for immunizing a non-human animal;
xiv) for preparation of hybridoma;
xv) in a method according to any one of preceding claims;
xvi) for use according to any one of preceding i)-xv), wherein said use is in vivo and/or ex vivo and/or in vitro use;
xvii) for use according to any one of preceding i)-xvi), wherein said antigen is heterologous to the environment in which said antigen is used.

83. The use of the polymersome according to any one of preceding claims 1 to 52, wherein said polymersome having a diameter of about 100 nm or more and comprising a conjugated or attached antigen, wherein said conjugated or attached antigen is selected from the group consisting of:
i) a polypeptide;
ii) a carbohydrate;
iii) a polynucleotide, or
iv) a combination of i) and/or ii) and/or iii)
for eliciting an immune response.

84. The use of claim 83, wherein the diameter of the polymersome is in the range of from about 100 nm to 1 μm, or from about 140 nm to about 1 μm, or from about 140 nm to about 750 nm, or from about 140 nm to about 500 nm, or from about 140 nm to about 250 nm, from about 140 nm to about 240 nm, from about 150 nm to about 235 nm, from about 170 nm to about 230 nm, or from about 220 nm to about 180 nm, or from about 190 nm to about 210 nm.

85. The use of a collection of polymersomes as defined in any one of claims 1 to 52, having a mean diameter of about 100 nm or more, 110 nm or more, 120 nm or more, 130 nm or more, 140 nm or more, the polymersomes of the collection comprising a conjugated or attached antigen, wherein said conjugated or attached antigen is selected from the group consisting of:
i) a polypeptide;
ii) a carbohydrate;
iii) a polynucleotide, or
iv) a combination of i) and/or ii) and/or iii)
for eliciting an immune response.

86. The use of claim 83, wherein the diameter of the polymersome is in the range of from about 100 nm to 1 μm, or from about 140 nm to about 1 μm, or from about 140 nm to about 750 nm, or from about 140 nm to about 500 nm, or from about 140 nm to about 250 nm, from about 140 nm to about 240 nm, from about 150 nm to about 235 nm, from about 170 nm to about 230 nm, or from about 220 nm to about 180 nm, or from about 190 nm to about 210 nm.

87. The use according to any one of claims 83 to 86, wherein the polymersome is selected from a group consisting of: cationic, anionic and nonionic polymersome.

88. The use according to any one of claims 83 to 87, wherein the polymersome has a circumferential membrane (formed by) of an amphiphilic polymer.

89. The use of a polymersome according to claim 88, wherein the amphiphilic polymer is essentially non-immunogenic or essentially non-antigenic.

90. The use of a polymersome according to claim 87 or 88, wherein the amphiphilic polymer is neither an immunostimulant nor adjuvant.

91. The use of a polymersome according to any of claims 88 to 90, wherein the amphiphilic polymer comprises a diblock or a triblock (A-B-A or A-B-C) copolymer.

92. The use of a polymersome according to any one of claims 88 to 91, wherein the amphiphilic polymer comprises a copolymer poly(N-vinylpyrrolidone)-b-PLA.

93. The use of a polymersome according to any one of claims 88 to 92, wherein the amphiphilic polymer comprises at least one monomer unit of a carboxylic acid, an amide, an amine, an alkylene, a dialkylsiloxane, an ether or an alkylene sulphide.

94. The use of a polymersome according to any one of claims 88 to 93, wherein the amphiphilic polymer is a polyether block selected from the group consisting of an oligo(oxyethylene) block, a poly(oxyethylene) block, an oligo(oxypropylene) block, a poly(oxypropylene) block, an oligo(oxybutylene) block and a poly(oxybutylene) block.

95. The use of a polymersome according to any one of claims 88 to 94, wherein the amphiphilic polymer is a poly(butadiene)-poly(ethylene oxide) (PB-PEO) diblock copolymer.

96. The use of a polymersome according to claim 95, wherein said PB-PEO diblock copolymer comprises 5-50 blocks PB and 5-50 blocks PEO.

97. The use of a polymersome according to any one of claims 88 to 96, wherein the amphiphilic polymer is a poly(lactide)-poly(ethylene oxide)/1-palmitoyl-2-oleoyl-sn-glycero-3-phospho-L-serine (PLA-PEO/POPC) copolymer, preferably said PLA-PEO/POPC has a ratio of 75 to 25 (e.g., 75/25) of PLA-PEO to POPC (e.g., PLA-PEO/POPC).

98. The use of a polymersome according to any one of claims 88 to 97, wherein said amphiphilic polymer is a poly(caprolactone)-poly(ethylene oxide)/1-palmitoyl-2-oleoyl-sn-glycero-3-phospho-L-serine (PCL-PEO/POPC) copolymer, preferably said PCL-PEO/POPC has a ratio of 75 to 25 (e.g., 75/25) of PCL-PEO to POPC (e.g., PCL-PEO/POPC).

99. The use of a polymersome according to any one of claims 88 to 98, wherein said amphiphilic polymer is polybutadiene-polyethylene oxide (BD).

100. The use of polymersome according to any one of claims 88 to 99, wherein said polymersome comprises diblock copolymer PBD₂₁-PEO₁₄ (BD21) and/or the triblock copolymer PMOXA₁₂-PDMS₅₅-PMOXA₁₂.

101. The polymersome according to any one of the preceding claims 1 to 52, wherein the polymersome further comprises an encapsulated antigen.


SEQUENCE LISTING (5 sequences)

SEQ ID NO: 1 - protein, 386 aa, Gallus gallus
  MISC_FEATURE (1)..(386): Ovalbumin, UniProtKB Accession Number P01012
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 566 aa, Influenza A virus
  MISC_FEATURE (1)..(566): Influenza A virus (A/New York/38/2016(H1N1)) Hemagglutinin, UniProtKB Accession Number A0A192ZYK0
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 3 - protein, 566 aa, Influenza A virus
  MISC_FEATURE (1)..(566): Influenza A virus (A/swine/4/Mexico/2009(H1N1)) Hemagglutinin, UniProtKB Accession Number D2CE65
  misc_feature (239)..(240): Xaa can be any naturally occurring amino acid
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPPTSADQQSLYQNADTYVFVGSSRYSKKFKPEIAIRPKVRXXEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 4 - protein, 565 aa, Influenza A virus
  MISC_FEATURE (1)..(565): Influenza A virus (H1N1/A/Puerto rico/8/1934) Hemagglutinin
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 5 - protein, 566 aa, Influenza A virus
  MISC_FEATURE (1)..(566): Influenza A virus (A/California/07/2009(H1N1)) Hemagglutinin
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI